(19) United States
(12) Patent Application Publication
(10) Pub. No.: US 2016/0135734 A1
(43) Pub. Date: May 19, 2016

(54) COMBINATION THERAPY FOR SLEEP DISORDERED BREATHING AND HEART FAILURE

(71) Applicant: ResMed Limited, Bella Vista (AU)

(72) Inventor: Klaus Henry Schindhelm, Sydney (AU)

(21) Appl. No.: 14/944,626

(22) Filed: Nov. 18, 2015

Related U.S. Application Data
(60) Provisional application No. 62/081,758, filed on Nov. 19, 2014.

(51) Int. Cl. A61B 5/00; A61M 16/00; A61N 1/36; A61B 5/0205

(52) U.S. Cl. CPC A61B 5/4818; A61B 5/4848; A61B 5/0205; A61M 16/0069; A61B 5/4839; A61N 1/3601; A61N 1/36135

(57) ABSTRACT

Methods and apparatus provide evaluation of a cardiac condition of a patient with sleep disordered breathing. One or more processors, such as processors associated with any of ICDs, CRTs and/or respiratory pressure therapy devices, may be configured to receive data representing one or more respiratory parameters for the patient. The processor(s) may receive cardiac-related patient data for the patient. The processor(s) may determine a presence of a smooth hemodynamic baseline from the received data representing one or more respiratory parameters for the patient. The processors may evaluate the cardiac condition of the patient from the received cardiac-related patient data based on the determined presence of the smooth hemodynamic baseline.

### 1 CROSS-REFERENCE TO RELATED APPLICATIONS

This application claims the benefit of U.S. Provisional Application No. 62/081,758, filed 19 Nov. 2014, the entire disclosure of which is hereby incorporated herein by reference.

### 2 STATEMENT REGARDING FEDERALLY SPONSORED RESEARCH OR DEVELOPMENT

Not Applicable

### 3 THE NAMES OF PARTIES TO A JOINT RESEARCH DEVELOPMENT

Not Applicable

### 4 SEQUENCE LISTING

Not Applicable

### 1 BACKGROUND

1.1 Field of the Invention

The present technology relates to one or more of the detection, diagnosis, treatment, prevention and amelioration of respiratory-related disorders in connection with heart failure. In particular, the present technology relates to medical devices or apparatus, and their use.

1.2 Description of the Related Art

### 1.2.1 Human Respiratory System, Cardiovascular System, and Their Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the air into the venous blood and carbon dioxide to move out. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2011.

A range of respiratory disorders exist. Some examples of respiratory disorders include: Obstructive Sleep Apnea (OSA), Cheyne Stokes Respiration (CSR), Central Sleep Apnea (CSA), Obesity Hyperventilation Syndrome (OHS), Chronic Obstructive Pulmonary Disease (COPD), Neuromuscular Disease (NMD) or chest wall disorders. Otherwise healthy individuals may take advantage of systems and devices to prevent respiratory disorders from arising.

The cardiovascular system facilitates the gas exchange described above through the circulation of blood. A range of cardiovascular disorders exist, many of which relate to disorders of the heart. For example, heart failure occurs when the heart is unable to properly or efficiently pump blood through the heart, so that it may be circulated throughout the body. Heart failure may be acute or chronic in nature.

### 1.2.2 Therapies

Continuous Positive Airway Pressure (CPAP) therapy has been used to treat OSA. The mechanism of action is that continuous positive airway pressure acts as a pneumatic splint and may prevent upper airway occlusion by pushing the soft palate and tongue forward and away from the posterior oropharyngeal wall.

Non-invasive ventilation (NIV) provides ventilatory support to a patient through the upper airways to assist the patient in taking a full breath and/or maintain adequate oxygen levels in the body by doing some or all of the work of breathing. The ventilatory support is provided via a non-invasive patient interface. NIV has been used to treat CSR, OHS, COPD, NMD and chest wall disorders.

Invasive ventilation (IV) provides ventilatory support to patients that are no longer able to effectively breathe themselves and may be provided using a tracheostomy tube.

Ventilators may control the timing and pressure of breaths delivered to the patient and monitor the breaths taken by the patient. The methods of control and monitoring patients typically include volume-cycled and pressure-cycled methods. The volume-cycled methods may include among others, Pressure-Regulated Volume Control (PRVC), Volume Ventilation (VV), and Volume Controlled Continuous Mandatory Ventilation (VC-CMV) techniques. The pressure-cycled methods may involve, among others, Assist Control (AC), Synchronized Intermittent Mandatory Ventilation (SIMV), Controlled Mechanical Ventilation (CMV), Pressure Support Ventilation (PSV), Continuous Positive Airway Pressure (CPAP), or Positive End Expiratory Pressure (PEEP) techniques.

With regard to heart failure, cardiac treatments may include pharmaceutical drugs and cardiac resynchronization therapies (“CRTs”). In some instances, implantable cardiac devices, such as a pacemaker or implantable cardioverter-defibrillator (“ICD”), may be used to increase the heart's efficiency or to otherwise regulate the heart's pumping.

### 1.2.3 Treatment Systems

A treatment system may comprise a Respiratory Pressure Therapy Device (RPT device), an air circuit, a humidifier, and a patient interface. A treatment system may also include an implantable device. The implantable device may provide cardiac treatment, such as CRT.

### 1.2.3.1 Patient Interface

A patient interface may be used to interface respiratory equipment to its user, for example by providing a flow of air to an entrance to an airway of the user. The flow of air may be provided via a mask to the nose and/or mouth, a tube to the mouth or a tracheostomy tube to the trachea of the user. Depending upon the therapy to be applied, the patient interface may form a seal, e.g. with a face region of the patient, to facilitate the delivery of gas at a pressure at sufficient variance with ambient pressure to effect therapy, e.g. a positive pressure of about 10 cm H₂O. For other forms of therapy, such as the delivery of oxygen, the patient interface may not include a seal sufficient to facilitate delivery to the airways of a supply of gas at a positive pressure of about 10 cm H₂O.

### 1.2.3.2 Respiratory Pressure Therapy (RPT) Device

One known RPT device used for treating sleep disordered breathing is the S9 Sleep Therapy System, manufactured by ResMed. Another example of an RPT device is a ventilator. Ventilators such as the ResMed Stellar™ Series of Adult and Paediatric Ventilators may provide support for invasive and non-invasive non-dependent ventilation for a range of patients for treating a number of conditions such as but not limited to NMD, OHS and COPD. RPT devices have also been known as flow generators.

The ResMed Elisée™ 150 ventilator and ResMed VS III™ ventilator may provide support for invasive and non-invasive dependent ventilation suitable for adult or paediatric patients for treating a number of conditions. These ventilators provide volumetric and barometric ventilation modes with a single or double limb circuit.

RPT devices typically comprise a pressure generator, such as a motor-driven blower or a compressed gas reservoir, and are configured to supply a flow of air to the airway of a patient. In some cases, the flow of air may be supplied to the airway of the patient at positive pressure. The outlet of the RPT device is connected via an air circuit to a patient interface such as those described above.

RPT devices typically also include an inlet filter, various sensors and a microprocessor-based controller. A blower may include a servo-controlled motor, a volute and an impeller. In some cases a brake for the motor may be implemented to more rapidly reduce the speed of the blower so as to overcome the inertia of the motor and impeller. The braking can permit the blower to more rapidly achieve a lower pressure condition in time for synchronization with expiration despite the inertia. In some cases the pressure generator may also include a valve capable of discharging generated air to atmosphere as a means for altering the pressure delivered to the patient as an alternative to motor speed control. The sensors measure, amongst other things, motor speed, mass flow rate and outlet pressure, such as with a pressure transducer or the like. The controller may include data storage capacity with or without integrated data retrieval and display functions.

### 1.2.3.3 Humidifier

Delivery of a flow of air without humidification may cause drying of airways. Medical humidifiers are used to increase humidity and/or temperature of the flow of air in relation to ambient air when required, typically where the patient may be asleep or resting (e.g. at a hospital). As a result, a medical humidifier is preferably small for bedside placement, and it is preferably configured to only humidify and/or heat the flow of air delivered to the patient without humidifying and/or heating the patient's surroundings. Room-based systems (e.g. a sauna, an air conditioner, an evaporative cooler), for example, may also humidify air that is breathed in by the patient, however they would also humidify and/or heat the entire room, which may cause discomfort to the occupants.

The use of a humidifier with a flow generator or RPT device and the patient interface produces humidified gas that minimizes drying of the nasal mucosa and increases patient airway comfort. In addition, in cooler climates warm air applied generally to the face area in and about the patient interface is more comfortable than cold air.

### 1.2.3.4 Implantable Device

An implantable device is typically a medical device that replaces or supports a human organ. Typically, an implant will internally contact the body and may include some electronic control. For example, an implantable cardioverter-defibrillator (ICD) may be implanted in the chest or abdomen. The ICD may be configured to provide a cardiac treatment for arrhythmia. Such a device may provide electrical pulses or shocks (e.g., defibrillation) to help control arrhythmias, such as arrhythmias associated with sudden cardiac arrest. A pacemaker may provide a temporary pacing therapy such as for symptomatic bradyarrhythmias. Pacemakers may employ low energy electrical pulses for influencing cardiac rhythm. In some cases, such as for heart failure patients, a cardiac resynchronization therapy (CRT) device may be implanted. A CRT device is typically configured to pace both ventricles of the heart concurrently. A CRT device may also be a defibrillator. An implantable device may be configured to provide nerve stimulation, such as stimulation of a patient's hypoglossal nerve and/or phrenic nerve.

### 2 BRIEF SUMMARY OF THE TECHNOLOGY

The present technology may include methods for evaluating cardiac condition of a patient, such as a patient with sleep disordered breathing. In some cases, the present technology may include an apparatus or system for evaluating a cardiac condition of a patient, such as a patient with sleep disordered breathing.

Some versions of the present technology include a method for evaluating a cardiac condition of a patient with sleep disordered breathing. The method may include receiving data representing one or more respiratory parameters for the patient. The method may include receiving cardiac-related patient data for the patient. The method may include determining in a processor a presence of a smooth hemodynamic baseline from the received data representing one or more respiratory parameters for the patient. The method may include evaluating, with the processor, the cardiac condition of the patient from the received data representing one or more hemodynamic parameters for the patient based on the determined presence of the smooth hemodynamic baseline.

In some cases of the method, the determining may include detecting absence of sleep disordered breathing events for a predetermined period of time. The determining may involve detecting sleep disordered breathing events. The determining may also involve controlling a respiratory therapy to alleviate the detected sleep disordered breathing events for a predetermined period of time. In some cases, the method may further include collecting, with one or more sensors, cardiac-related patient data while the patient is within the smooth hemodynamic baseline. Optionally, the evaluating may be based on the cardiac-related patient data. The controlling of the method may include controlling a respiratory pressure therapy apparatus to deliver respiratory pressure therapy for the patient. Optionally, the respiratory pressure therapy may involve an adaptive servo-ventilation therapy. The sleep disordered breathing events may include one or more of apnea, hypopnea, Cheyne-Stokes breathing, snoring and flow limitation.

In some cases of the method, the controlling may involve controlling an implanted device to provide at least one of genioglossal nerve stimulation and phrenic nerve stimulation. The method may also include determining, based on the evaluating, efficacy of a cardiac treatment that has been provided to the patient. The cardiac treatment may include one or more of administering a pharmaceutical cardiac drug and application of a cardiac resynchronization therapy. The method may further include titrating the cardiac treatment according to the hemodynamic parameters. The titration of the cardiac treatment may include determination of a diuretic dose. The titration of the cardiac treatment may include an adjustment to a target ventilation of a respiratory pressure therapy. The respiratory pressure therapy may be a pressure support ventilation therapy controlled in accordance with the target ventilation.

In some cases of the method, the evaluating may include outputting one or more hemodynamic parameters according to a time period of the smooth hemodynamic baseline. The evaluating may include detecting a change in one or more hemodynamic parameters within a time period initiated with the smooth hemodynamic baseline. Optionally, the received data representing one or more hemodynamic parameters may include measurements generated by a pulmonary artery sensor. The received data representing one or more respiratory parameters may include measurements generated by a Doppler radar motion sensor.

Some versions of the present technology include a system for evaluating a cardiac condition of a patient with sleep disordered breathing. The system may include one or more memories. The system may include one or more processors in communication with the one or more memories. The one or more processors may be configured to receive data representing one or more respiratory parameters for the patient. The one or more processors may be configured to receive data representing cardiac-related patient data for the patient. The one or more processors may be configured to determine a presence of a smooth hemodynamic baseline from the received data representing one or more respiratory parameters for the patient. The one or more processors may be configured to evaluate the cardiac condition of the patient from the received data representing cardiac-related patient data for the patient based on the determined presence of the smooth hemodynamic baseline.

In some versions of the system, determination of the presence of the smooth hemodynamic baseline may involve detecting absence of sleep disordered breathing events for a predetermined period of time. Determination of the presence of the smooth hemodynamic baseline may include detecting sleep disordered breathing events and controlling a respiratory therapy to alleviate the detected sleep disordered breathing events for a predetermined period of time. The system may further collect, with one or more sensors, cardiac-related patient data while the patient is within the smooth hemodynamic baseline. In some cases, evaluation of the cardiac condition by the system may be based on the cardiac-related patient data.

Optionally, the system may include a respiratory pressure therapy apparatus configured to alleviate detected sleep disordered breathing events by controlling a respiratory pressure therapy for the patient. The respiratory pressure therapy may include an adaptive servo-ventilation therapy. The sleep disordered breathing events may include one or more of apnea, hypopnea, Cheyne-Stokes breathing, snoring and flow limitation. The system may include an implanted device configured to alleviate sleep disordered breathing events by controlling providing at least one of genioglossal nerve stimulation and phrenic nerve stimulation.

Optionally, the one or more processors of the system may be further configured to determine, based on the evaluating, efficacy of a cardiac treatment that has been provided to the patient. The cardiac treatment may include one or more of administering a pharmaceutical cardiac drug and providing a cardiac resynchronization therapy. The evaluation may further include titration of the cardiac treatment according to the hemodynamic parameters. The cardiac treatment may include a diuretic dose and the titration may involve determining the diuretic dose. The titration of the cardiac treatment may include an adjustment to a target ventilation of a respiratory pressure therapy. The respiratory pressure therapy may be a pressure support ventilation therapy controlled in accordance with the target ventilation and may be provided by an adaptive servo-ventilator of the system.

In some cases, the evaluation may include outputting, by the one or more processors, one or more hemodynamic parameters according to a time period of the smooth hemodynamic baseline. The evaluation of the system may include detecting by the one or more processors a change in one or more hemodynamic parameters within a time period initiated with the smooth hemodynamic baseline. The received data representing one or more hemodynamic parameters may include measurements generated by a pulmonary artery sensor. The received data representing one or more respiratory parameters may include measurements generated by a Doppler radar motion sensor.

Some versions of the present technology include an apparatus, which may optionally be part of the system, for evaluating a cardiac condition of a patient with sleep disordered breathing. The apparatus may include a flow generator for providing a supply of air to a patient interface at variable pressures. The flow generator may be adapted for coupling with a patient interface for delivering the supply of air to an entrance of an airway of the patient. The apparatus may include one or more sensors configured to sense one or more respiratory parameters for the patient. The apparatus may include one or more processors. The one or more processors may be configured to control the supply of air at variable pressures. The one or more processors may be configured to receive data representing the one or more respiratory parameters for a patient. The one or more processors may be configured to determine a presence of a smooth hemodynamic baseline from the received data representing the one or more respiratory parameters for the patient.

In some versions, the apparatus may further include one or more sensors configured to sense one or more hemodynamic parameters for the patient. The one or more processors may be further configured to receive data representing one or more hemodynamic parameters for the patient, and to evaluate the cardiac condition of the patient from the received data representing one or more hemodynamic parameters for a patient based on the determined presence of the smooth hemodynamic baseline. The one or more processors may be further configured to receive a transmission from an implanted device. The one or more processors may be further configured to receive a transmission from a Doppler radar motion sensor. The one or more processors may be further configured to change a setting for control of the supply of air based on the received transmission. Optionally, the control of the supply of air at variable pressures by the apparatus may be adaptive servo-ventilation.

Some versions of the present technology include a method for evaluating a cardiac condition of a patient with sleep disordered breathing. The method may include controlling a flow generator to provide a supply of air to a patient interface. The flow generator may be adapted for coupling with a patient interface for delivering the supply of air to an entrance of an airway of the patient. The method may include sensing, with one or more sensors, one or more respiratory parameters for the patient. The method may further include, in one or more processors: controlling the supply of air at variable pressures; receiving data representing the one or more respiratory parameters for a patient; and determining a presence of a smooth hemodynamic baseline from the received data representing the one or more respiratory parameters for the patient.

In some versions, the method may include sensing, with one or more sensors, one or more hemodynamic parameters for the patient. The method may include, in the one or more processors, receiving data representing one or more hemodynamic parameters for the patient; and evaluating the cardiac condition of the patient from the received data representing one or more hemodynamic parameters for a patient based on the determined presence of the smooth hemodynamic baseline. The method may include with the one or more processors, receiving a transmission from an implanted device. The method may include changing a setting for control of the supply of air based on the received transmission. The controlling the supply of air at variable pressures may include adaptive servo-ventilation.

In some cases, the method may include, with the one or more processors, receiving a transmission from a Doppler radar motion sensor. The method may include, with the one or more processors, changing a setting for control of the supply of air based on the received transmission. The controlling the supply of air at variable pressures may include adaptive servo-ventilation.

Apparatus and methods described heretofore and herein provide technological solutions to help improve monitoring and/or treatment of patients with cardiac conditions such as in conjunction with disordered breathing. Aspects of the methods and apparatus described can provide improvements in the functioning of processors for such devices, including for example, ICDs, CRTs, respiratory pressure therapy devices and/or servers or other processing devices that may communicate with such devices or generate analytical information from an analysis of automated measurements. The methods and apparatus also provide improvements in the technical field of cardiac treatment devices and/or cardiac monitoring devices that may be useful for improving or maintaining the condition of one or more cardiac patients.

Of course, portions of the aspects may form sub-aspects of the present technology. Also, various ones of the sub-aspects and/or aspects may be combined in various manners and also constitute additional aspects or sub-aspects of the present invention.

Other features of the technology will be apparent from consideration of the information contained in the following detailed description, abstract, drawings and claims.

### 3 BRIEF DESCRIPTION OF THE SEVERAL VIEWS OF THE DRAWINGS

The present technology is illustrated by way of example, and not by way of limitation, in the figures of the accompanying drawings, in which like reference numerals refer to similar elements including:

3.1 Treatment Systems

FIG. 1A shows a portion of the system in accordance with one form of the present technology. A patient 1000 wearing a patient interface 3000, in the form of a full-face mask (shown in phantom), receives a supply of air at positive pressure from an RPT device 4000. Air from the RPT device is humidified in a humidifier 5000, and passes along an air circuit 4170 to the patient 1000.

FIG. 1B shows an implanted portion of the system including patient 1000 having an implanted device 6000.

FIG. 1C shows a portion of the system in accordance with one form of the present technology. A patient 1000 is being monitored by an external sensor 7000.

3.2 Respiratory System

FIG. 2 shows an overview of a human respiratory system including the nasal and oral cavities, the larynx, vocal folds, oesophagus, trachea, bronchus, lung, alveolar sacs, heart and diaphragm.

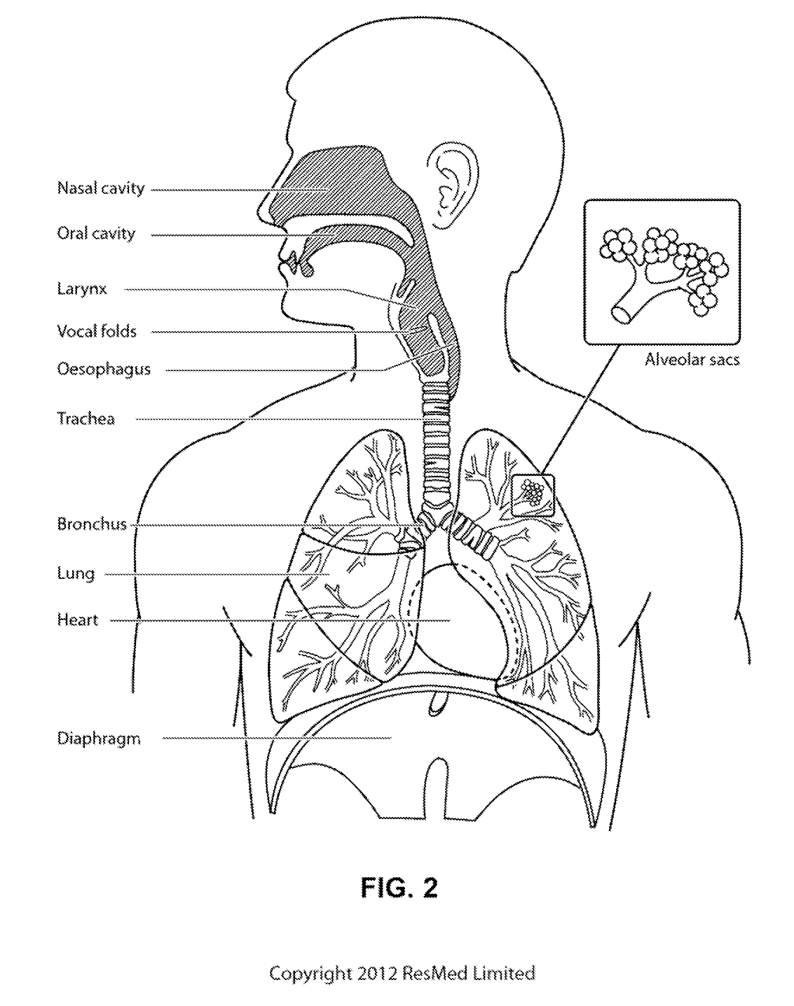

3.3 Patient Interface

FIG. 3 shows an example of a non-invasive patient interface.

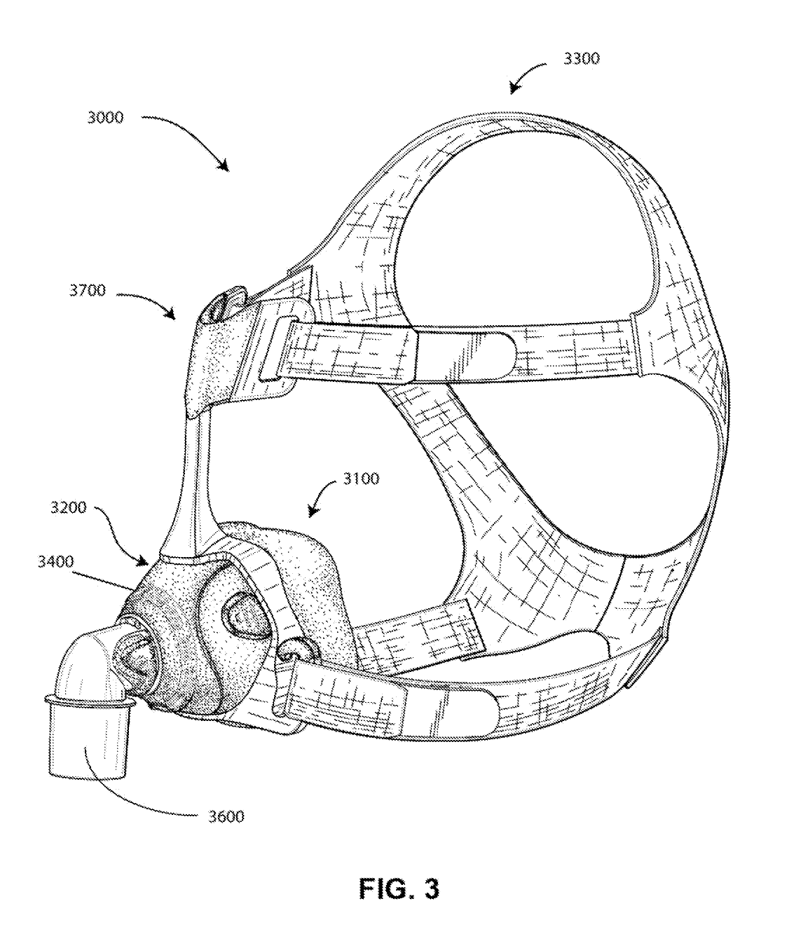

3.4 Respiratory Pressure Therapy (RPT) Device

FIG. 4A shows an RPT device in accordance with one form of the present technology.

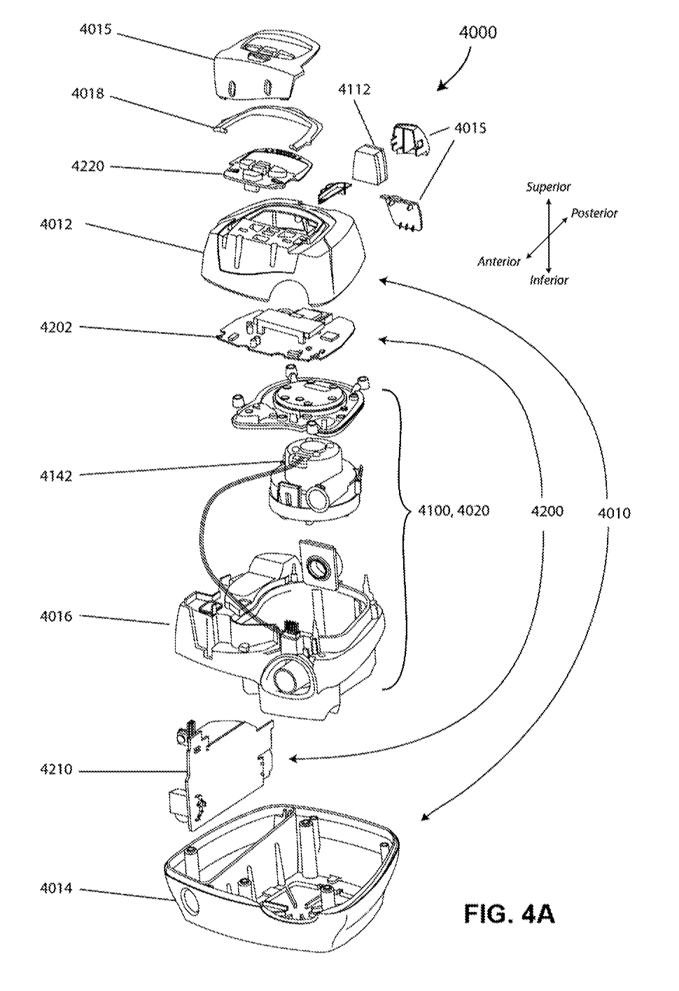

FIG. 4B shows a schematic diagram of the pneumatic circuit of the RPT device of FIG. 4A. The directions of upstream and downstream are indicated.

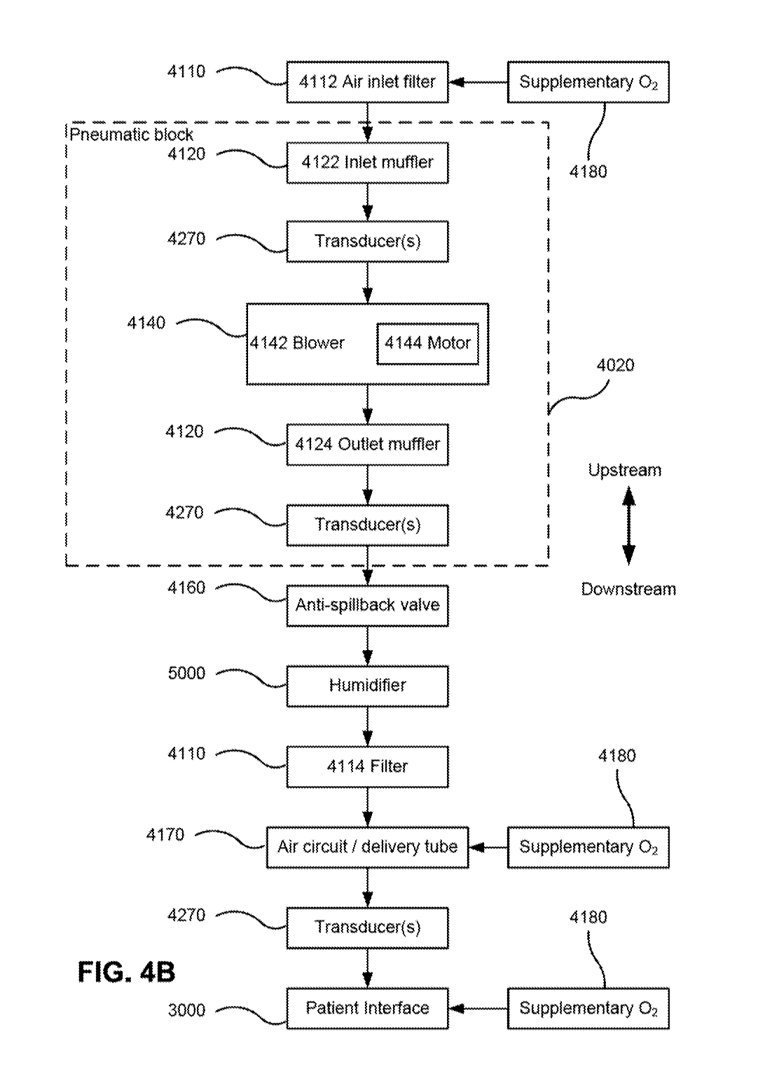

FIG. 4C shows a schematic diagram of the electrical components of the RPT device of FIG. 4A.

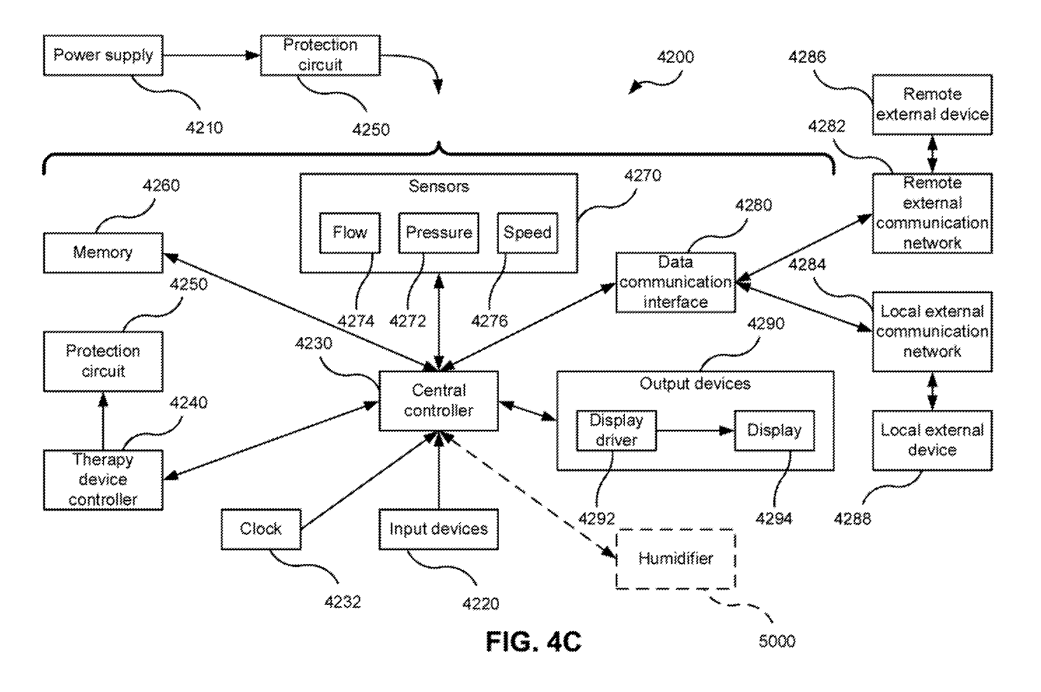

3.5 Humidifier

FIG. 5 shows a humidifier in accordance with one form of the present technology.

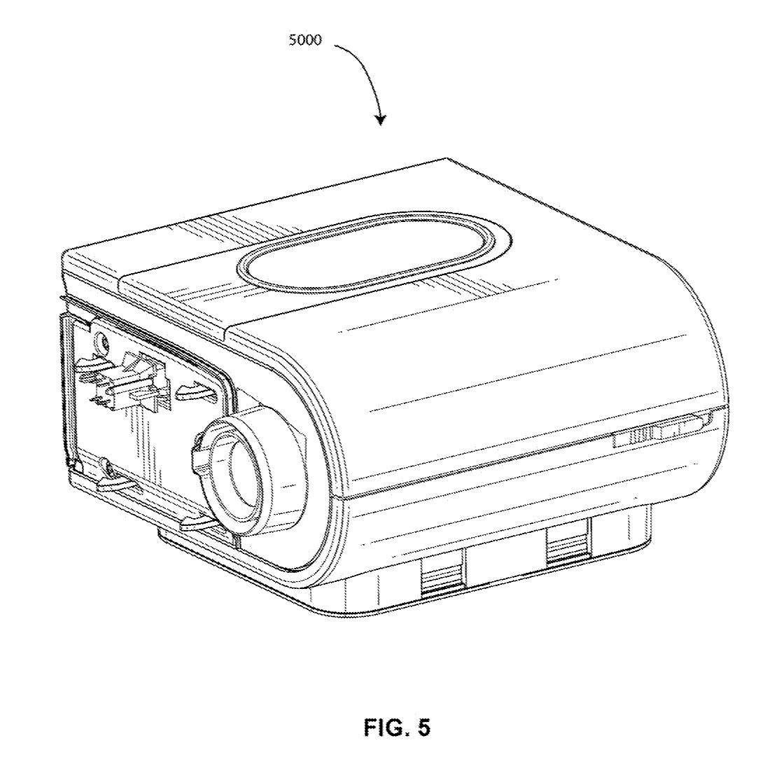

3.6 Breathing Waveforms

FIG. 6 shows a model typical breath waveform of a patient while sleeping, the horizontal axis is time, and the vertical axis is respiratory flow.

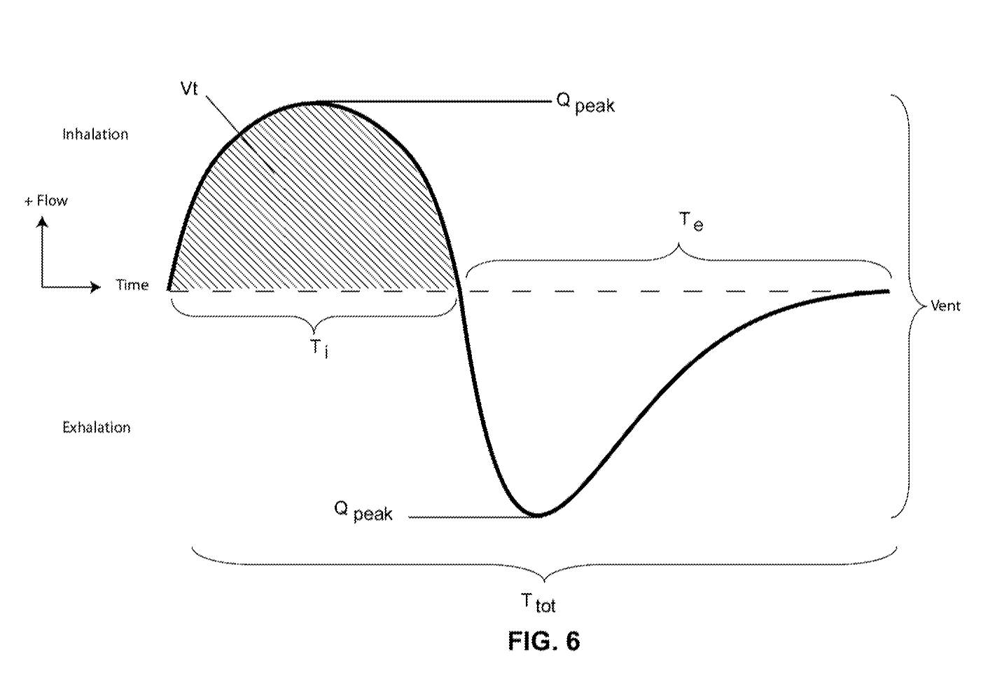

3.7 Combination Therapy System

FIG. 7 shows an example system 700 in which aspects of the present technology may be implemented.

FIG. 8 shows a flow diagram 800 of operations that may be performed with patient devices disclosed herein in connection with the collection and transmission of patient data.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

4.1 Treatment Systems

In one form, the present technology concerns a system for a combined treatment of respiratory and cardiac disorders. The system may involve both implanted devices and non-implanted devices. The non-implanted devices may include an RPT device 4000 for providing respiratory pressure therapy to the patient 1000 such as via an air circuit 4170 leading to a patient interface 3000.

The implanted device may be configured to implement respiratory therapy through the use of nerve stimulation, as well as cardiac treatment, such as by performing cardiac resynchronization therapy. The implanted device may be configured to also collect and transmit data relating to the patient's condition. In particular, the implanted device may, for example, monitor the patient's cardiac output, such as by measuring the heart rate, arterial pressure, ventricle timing, stroke volume, and other parameters relating to the heart's performance.

4.2 Therapy

In an aspect of the present technology, a patient's sleep disordered breathing (“SDB”) may be treated in combination with treatment of a cardiac condition. The resulting treatment may provide a synergistic benefit to the patient. For example, a patient may have been prescribed a treatment for a cardiac condition, such as being prescribed a pharmaceutical drug for treating chronic or acute heart failure. In order to determine the efficacy of the prescribed cardiac treatment, the patient's cardiac condition may be monitored, such as by measuring the patient's heart rate, blood pressure, blood flow, ventricular contractions, or any other factors that may be affected by the cardiac condition and related treatment. A system of the present technology may collect these cardiac-related measurements throughout the day, including while the patient is sleeping. The cardiac-related measurements may then be analyzed by the system to determine if the prescribed cardiac treatment is performing effectively.

A patient's SDB may affect one or more of these cardiac-related measurements. For example, a patient may experience sudden fluctuations in heart rate and blood pressure during the occurrence of sleep apnea or Cheyne-Stokes respiration. Accordingly, the cardiac-related measurements may not relate solely to the efficacy of the prescribed cardiac treatment, as there are times when a patient's SDB may impact causation of particular cardiac events. SDB therefore may prevent or obfuscate a proper analysis of the prescribed cardiac treatment.

The present technology may be implemented to permit cardiac-related measurements to be taken or identified at a particular time, such as by isolating particular cardiac-related measurements attributable to the patient's cardiac condition from respiratory events, such as SDB. Such a system may determine that a patient has maintained some predetermined respiratory event-free baseline (e.g., a confirmed absence of SDB events for a predetermined period of time) before making or evaluating one or more cardiac-related measurements. Such a system may assist the patient in maintaining the predetermined baseline by applying one or more treatments to address or alleviate the patient's SDB. For example, the baseline may be maintained by the controlled application of respiratory pressure therapy at the entrance of the patient's airway and/or through the controlled application of respiratory-related nerve stimulation. Such a baseline may be referred to herein as a smooth hemodynamic baseline as it attempts to isolate the patient's SDB from impacting cardiac performance and blood flow so that cardiac-related measurements may be suitably isolated from other potential affecting factors. Examples of these methods are described in more detail herein.

4.3 Respiratory Pressure Therapy

### 4.3.1 Patient Interface

A non-invasive patient interface 3000 in accordance with one form of the present technology comprises the following functional aspects: a seal-forming structure 3100, a plenum chamber 3200, a positioning and stabilising structure 3300, a vent 3400, a connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to facilitate the supply of air at positive pressure to the airways.

### 4.3.2 RPT Device

An example RPT device 4000 that may be suitable for implementing aspects of the present technology may include mechanical and pneumatic components 4100, electrical components 4200 and may be programmed to execute one or more of the control methodologies or algorithms described throughout this specification. The RPT device may have an external housing 4010, preferably formed in two parts, an upper portion 4012 of the external housing 4010, and a lower portion 4014 of the external housing 4010. In alternative forms, the external housing 4010 may include one or more panel(s) 4015. Preferably the RPT device 4000 comprises a chassis 4016 that supports one or more internal components of the RPT device 4000. In one form a pneumatic block 4020 is supported by, or formed as part of the chassis 4016. The RPT device 4000 may include a handle 4018.

The pneumatic path of the RPT device 4000 preferably comprises an inlet air filter 4112, an inlet muffler 4122, a controllable pressure generator 4140 capable of supplying air at positive pressure (preferably a blower 4142), and an outlet muffler 4124. One or more pressure sensors 4272 and flow rate sensors 4274 are included in the pneumatic path and may include or receive data via communication from any additional sensor devices, such as any of the sensors described throughout this specification.

The preferred pneumatic block 4020 comprises a portion of the pneumatic path that is located within the external housing 4010.

The RPT device 4000 preferably has an electrical power supply 4210, one or more input devices 4220, a central controller 4230, a therapy device controller 4240 and/or any of the controllers previously described, a pressure generator 4140, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the RPT device 4000 may include more than one PCBA 4202.

The central controller 4230 of the RPT device 4000, which may include one or more processors, can be programmed to execute one or more algorithm modules, preferably including a pre-processing module, a therapy engine module, a pressure control module, and further preferably a fault condition module. It may further include a vent control module that may be configured with one or more of the vent control methodologies described throughout this specification.

### 4.3.2.1 RPT Device Mechanical & Pneumatic Components

### 4.3.2.1.1 Air Filter(s)

An RPT device in accordance with one form of the present technology may include an air filter 4110, or a plurality of air filters 4110.

In one form, an inlet air filter 4112 is located at the beginning of the pneumatic path upstream of a pressure generator 4140.

In one form, an outlet air filter 4114, for example an antibacterial filter, is located between an outlet of the pneumatic block 4020 and a patient interface 3000.

### 4.3.2.1.2 Muffler(s)

An RPT device in accordance with one form of the present technology may include a muffler 4120, or a plurality of mufflers 4120.

In one form of the present technology, an inlet muffler 4122 is located in the pneumatic path upstream of a pressure generator 4140.

In one form of the present technology, an outlet muffler 4124 is located in the pneumatic path between the pressure generator 4140 and a patient interface 3000.

### 4.3.2.1.3 Pressure Generator

In a preferred form of the present technology, a pressure generator 4140 for producing a flow of air at positive pressure is a controllable blower 4142. For example the blower may include a brushless DC motor 4144 with one or more impellers housed in a volute. The blower may be capable of delivering a supply of air, for example about 120 litres/minute, at a positive pressure in a range from about 4 cm H₂O to about 20 cm H₂O, or in other forms up to about 30 cm H₂O.

The pressure generator 4140 is under the control of the therapy device controller 4240.

In other forms, a pressure generator 4140 may be a piston-driven pump, a pressure regulator connected to a high pressure source (e.g. compressed air reservoir), or a bellows.

### 4.3.2.1.4 Transducer(s)

In one form of the present technology, one or more transducers 4270 are located upstream of the pressure generator 4140. The one or more transducers 4270 are constructed and arranged to measure properties such as a flow rate, a pressure or a temperature at that point in the pneumatic path.

In one form of the present technology, one or more transducers 4270 are located downstream of the pressure generator 4140, and upstream of the air circuit 4170. The one or more transducers 4270 are constructed and arranged to measure properties of the air at that point in the pneumatic path.

In one form of the present technology, one or more transducers 4270 are located proximate to the patient interface 3000.

In one form, a signal from a transducer 4270 may be filtered, such as by low-pass, high-pass or band-pass filtering.

### 4.3.2.1.5 Anti-Spill Back Valve

In one form of the present technology, an anti-spill back valve 4160 is located between the humidifier 5000 and the pneumatic block 4020. The anti-spill back valve 4160 is constructed and arranged to reduce the risk that water will flow upstream from the humidifier 5000, for example to the motor 4144.

### 4.3.2.1.6 Air Circuit

An air circuit 4170 in accordance with one form of the present technology is constructed and arranged to allow a flow of air between the pneumatic block 4020 and the patient interface 3000.

### 4.3.2.1.7 Oxygen Delivery

In one form of the present technology, supplemental oxygen 4180 is delivered to a point in the pneumatic path.

In one form of the present technology, supplemental oxygen 4180 is delivered upstream of the pneumatic block 4020.

In one form of the present technology, supplemental oxygen 4180 is delivered to the air circuit 4170.

In one form of the present technology, supplemental oxygen 4180 is delivered to the patient interface 3000.

### 4.3.2.2 RPT Device Electrical Components

### 4.3.2.2.1 Power Supply

In one form of the present technology power supply 4210 is internal of the external housing 4010 of the RPT device 4000. In another form of the present technology, power supply 4210 is external of the external housing 4010 of the RPT device 4000.

In one form of the present technology power supply 4210 provides electrical power to the RPT device 4000 only. In another form of the present technology, power supply 4210 provides electrical power to both RPT device 4000 and humidifier 5000. The power supply may also optionally provide power to any actuator, controller and/or sensors for a vent arrangement as described throughout this specification

### 4.3.2.2.2 Input Devices

In one form of the present technology, an RPT device 4000 includes one or more input devices 4220 in the form of buttons, switches or dials to allow a person to interact with the device. These may be implemented for entering settings for operation of the components of the RPT device such as the vent arrangement. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230.

In one form the input device 4220 may be constructed and arranged to allow a person to select a value and/or a menu option.

### 4.3.2.2.3 Central Controller

In one form of the present technology, the central controller 4230 is a dedicated electronic circuit configured to receive input signal(s) from the input device 4220, and to provide output signal(s) to the output device 4290 and/or the therapy device controller 4240.

In one form, the central controller 4230 is an application-specific integrated circuit. In another form, the central controller 4230 comprises discrete electronic components.

In another form of the present technology, the central controller 4230 is a processor suitable to control an RPT device 4000 such as an x86 INTEL processor.

A processor of a central controller 4230 suitable to control an RPT device 4000 in accordance with another form of the present technology includes a processor based on ARM Cortex-M processor from ARM Holdings. For example, an STM32 series microcontroller from ST MICROELECTRONICS may be used.

Another processor suitable to control an RPT device 4000 in accordance with a further alternative form of the present technology includes a member selected from the family ARM9-based 32-bit RISC CPUs. For example, an STR9 series microcontroller from ST MICROELECTRONICS may be used.

In certain alternative forms of the present technology, a 16-bit RISC CPU may be used as the processor for the RPT device 4000. For example a processor from the MSP430 family of microcontrollers, manufactured by TEXAS INSTRUMENTS, may be used.

The processor is configured to receive input signal(s) from one or more transducers 4270, and one or more input devices 4220.

The processor is configured to provide output signal(s) to one or more of an output device 4290, a therapy device controller 4240, a data communication interface 4280 and humidifier controller 5250.

In some forms of the present technology, the processor of the central controller 4230, or multiple such processors, is configured to implement the one or more methodologies described herein such as the one or more algorithms expressed as computer programs stored in a non-transitory computer readable storage medium, such as memory 4260. In some cases, as previously discussed, such processor(s) may be integrated with an RPT device 4000. However, in some forms of the present technology the processor(s) may be implemented discretely from the flow generation components of the RPT device 4000, such as for purpose of performing any of the methodologies described herein without directly controlling delivery of a respiratory treatment. For example, such a processor may perform any of the methodologies described herein for purposes of determining control settings for a ventilator or other respiratory related events by analysis of stored data such as from any of the sensors described herein. Similarly, such a processor may perform any of the methodologies described herein for purposes controlling operation of any vent arrangement described in this specification.

### 4.3.2.2.4 Clock

The RPT device 4000 may include a clock 4232 that is connected to processor.

### 4.3.2.2.5 Therapy Device Controller

In one form of the present technology, therapy device controller 4240 is a pressure control module 4330 that forms part of the algorithms executed by the processor of the central controller 4230.

In one form of the present technology, therapy device controller 4240 is a dedicated motor control integrated circuit. For example, in one form a MC33035 brushless DC motor controller, manufactured by ONSEMI is used.

### 4.3.2.2.6 Protection Circuits

Preferably an RPT device 4000 in accordance with the present technology comprises one or more protection circuits 4250.

One form of protection circuit 4250 in accordance with the present technology is an electrical protection circuit.

One form of protection circuit 4250 in accordance with the present technology is a temperature or pressure safety circuit.

### 4.3.2.2.7 Memory

In accordance with one form of the present technology the RPT device 4000 includes memory 4260, preferably non-volatile memory. In some forms, memory 4260 may include battery powered static RAM. In some forms, memory 4260 may include volatile RAM.

Preferably memory 4260 is located on PCBA 4202. Memory 4260 may be in the form of EEPROM, or NAND flash.

Additionally or alternatively, RPT device 4000 includes removable form of memory 4260, for example a memory card made in accordance with the Secure Digital (SD) standard.

In one form of the present technology, the memory 4260 acts as a non-transitory computer readable storage medium on which is stored computer program instructions expressing the one or more methodologies described herein, such as the one or more algorithms.

### 4.3.2.2.8 Transducers

Transducers may be internal of the device, or external of the RPT device. External transducers may be located for example on or form part of the air delivery circuit, e.g. the patient interface. External transducers may be in the form of non-contact sensors such as a Doppler radar motion sensor that transmit or transfer data to the RPT device.

### 4.3.2.2.8.1 Flow Rate Sensor

A flow rate sensor 4274 in accordance with the present technology may be based on a differential pressure transducer, for example, an SDP600 Series differential pressure transducer from SENSIRION. The differential pressure transducer is in fluid communication with the pneumatic circuit, with one of each of the pressure transducers connected to respective first and second points in a flow restricting element.

In use, a signal representing total flow rate Qt from the flow rate sensor 4274 is received by the processor.

### 4.3.2.2.8.2 Pressure Sensor

A pressure sensor 4272 in accordance with the present technology is located in fluid communication with the pneumatic circuit. An example of a suitable pressure sensor is a sensor from the HONEYWELL ASDX series. An alternative suitable pressure sensor is a sensor from the NPA Series from GENERAL ELECTRIC.

In use, a signal from the pressure sensor 4272 is received by the central controller 4230. In one form, the signal from the pressure sensor 4272 is filtered prior to being received by the central controller 4230.

### 4.3.2.2.8.3 Motor Speed Transducer

In one form of the present technology a motor speed transducer 4276 is used to determine a rotational velocity of the motor 4144 and/or the blower 4142. A motor speed signal from the motor speed transducer 4276 is preferably provided to the therapy device controller 4240. The motor speed transducer 4276 may, for example, be a speed sensor, such as a Hall effect sensor.

### 4.3.2.2.9 Data Communication Interface

In one preferred form of the present technology, a data communication interface 4280 is provided, and is connected to central controller 4230. Data communication interface 4280 is preferably connectable to remote external communication network 4282. Data communication interface 4280 is preferably connectable to local external communication network 4284. Preferably remote external communication network 4282 is connectable to remote external device 4286. Preferably local external communication network 4284 is connectable to local external device 4288.

In one form, data communication interface 4280 is part of processor of central controller 4230. In another form, data communication interface 4280 is an integrated circuit that is separate from the central controller 4230.

In one form, remote external communication network 4282 is the Internet. The data communication interface 4280 may use wired communication (e.g. via Ethernet, or optical fibre) or a wireless protocol to connect to the Internet.

In one form, local external communication network 4284 utilises one or more communication standards, such as Bluetooth, or a consumer infrared protocol.

In one form, remote external device 4286 is one or more computers, for example a cluster of networked computers. In one form, remote external device 4286 may be virtual computers, rather than physical computers. In either case, such remote external device 4286 may be accessible to an appropriately authorised person such as a clinician.

Local external device 4288 may be a personal computer, mobile phone, tablet or remote control.

### 4.3.2.2.10 Output Devices Including Optional Display, Alarms

An output device 4290 in accordance with the present technology may take the form of one or more of a visual, audio and haptic unit. A visual display may be a Liquid Crystal Display (LCD) or Light Emitting Diode (LED) display.

### 4.3.2.2.10.1 Display Driver

A display driver 4292 receives as an input the characters, symbols, or images intended for display on the display 4294, and converts them to commands that cause the display 4294 to display those characters, symbols, or images.

### 4.3.2.2.10.2 Display

A display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol.

4.4 Implanted Device Therapy

In addition to, or alternatively to, the respiratory pressure therapy described above, the disclosed system may treat a patient's SDB through respiratory therapy, including genioglossal nerve stimulation and/or transvenous phrenic nerve stimulation. Genioglossal nerve stimulation involves transmitting an electrical stimulation to a patient's hypoglossal nerve. The stimulation of the hypoglossal nerve causes a contraction in the patient's genioglossus muscle, thereby producing an anterior displacement of the base of the patient's tongue. This contraction may then reduce any occlusion-causing pressure along the pharynx, thereby alleviating a patient's OSA or some other form of SDB.

Phrenic nerve stimulation involves transmitting an electrical stimulation to a patient's phrenic nerve so as to cause a contraction in the patient's diaphragm. By regulating the contraction of the diaphragm, phrenic nerve stimulation may regulate a patient's breathing and prevent a patient from experiencing CSA or other forms of SDB.

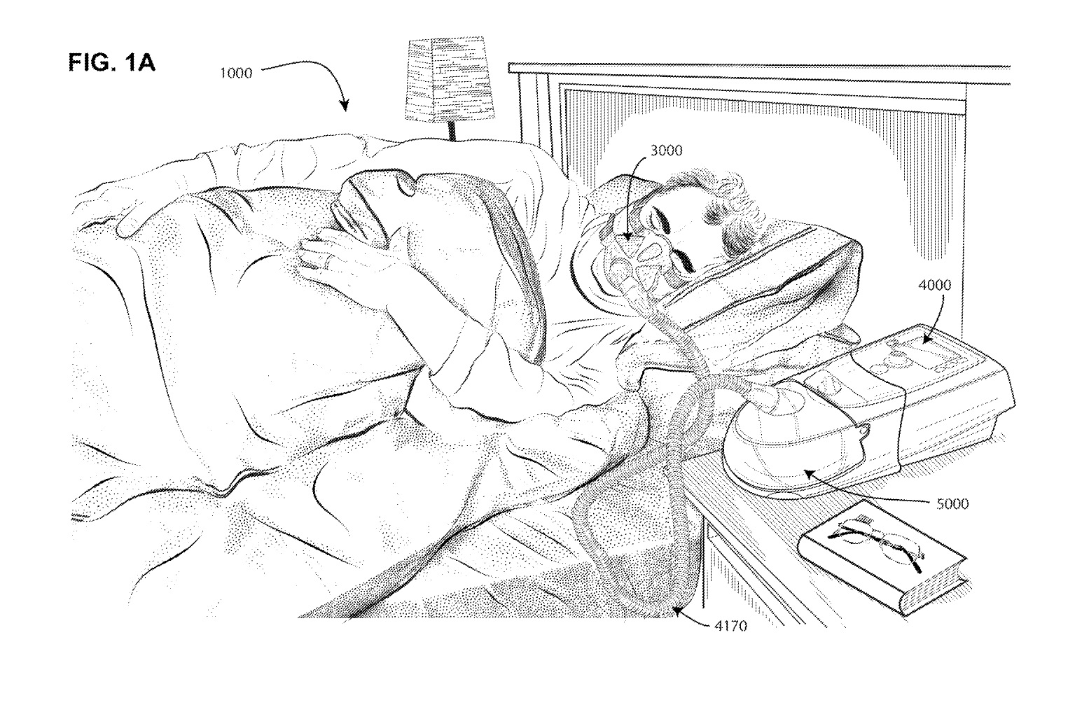

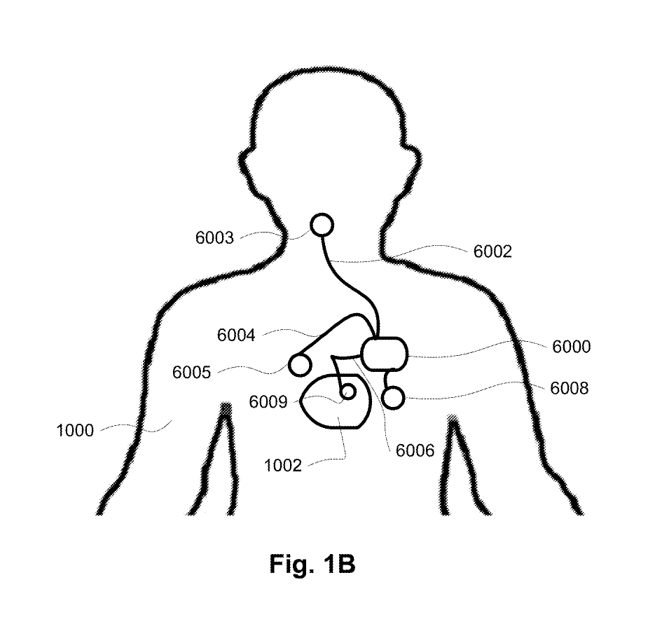

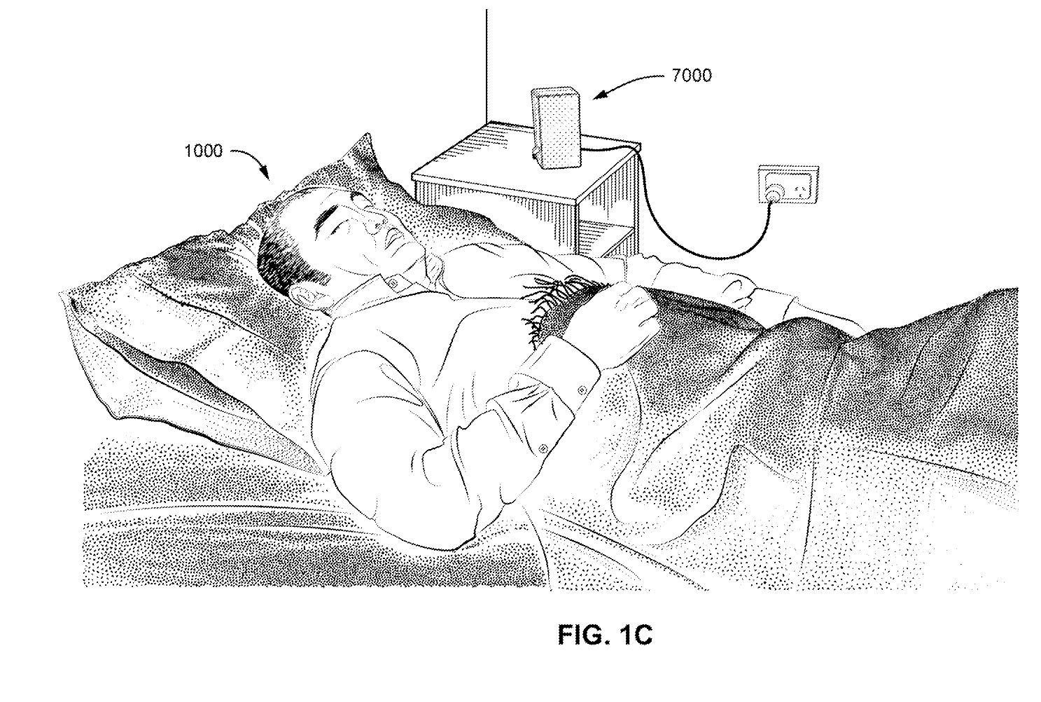

In accordance with one aspect of the disclosure, respiratory therapy including genioglossal nerve stimulation and/or transvenous phrenic nerve stimulation may be administered by a device that is implanted in the patient's body. For example, FIG. 1B shows example implanted device 6000, which may provide both cardiac and respiratory therapy to patient 1000. In particular, implanted device 6000 may provide genioglossal nerve stimulation to patient 1000 by transmitting electrical impulses along one or more wires, such as lead wire 6002, to one or more electrodes 6003 that are positioned on or around the genioglossal nerve of patient 1000. The intensity of the electrical pulse provided by implanted device 6000 may be controlled or adjusted. In particular, the intensity of the electrical pulse may be set, for example with a processor of the implanted device 6000 or a processor in communication with the implanted device 6000, so that the intensity is high enough to cause the desired contraction of the genioglossus muscle but low enough to avoid awakening the patient during sleep.

The electrical impulses may be provided in accordance with a predetermined protocol (e.g., algorithm carried out by a processor controlling the implanted device 6000) that prevents an obstruction from occurring during certain phases of the patient's breathing. If multiple electrodes 6003 are used, they may provide electrical impulses simultaneously or independently from one another in accordance with the predetermined protocol. For example, each electrode 6003 may provide electrical impulses that vary with respect to intensity and frequency.

Implanted device 6000 may also provide phrenic nerve stimulation by transmitting electrical impulses along lead wire 6004 to one or more electrode(s) 6005. Lead wire 6004 may be a transvenous lead that runs within a cardiophrenic vein near the patient's heart 1002. The transvenous electrode(s) 6005 may be placed within the patient so as to limit the electrode's effect on muscles that do not relate to respiration. The patient's phrenic nerve may also be accessed by running lead wire 6004 through a large vein, such as through the jugular or the superior vena cava.

Implanted device 6000 may also provide cardiac treatment to patient 1000. For example, implanted device 6000 may include a pacemaker or some other CRT or other ICD device. One or more lead wires 6006 may be used to provide electrical impulses to electrodes 6009 within the patient's heart 1002 so as to regulate the contraction of one or more muscles within heart 1002. In this way, the heartbeat of patient 1000 may be controlled so as to maintain a normal and efficient rhythm.

Lead wires 6002, 6004, and 6006 may optionally be coupled with one or more sensors (e.g., electrodes) for collecting cardiac-related patient data, such as the patient's pulse, heart rate, blood pressure, blood flow, ventricular contractions, etc. or any other hemodynamic parameters. In addition, implanted device 6000 may collect data related to the patient's respiratory system, such as the patient's breathing rate, blood oximetry levels, or carbon dioxide levels. In particular, one or more sensors 6008 may be placed in the proximity of the patient's lungs and diaphragm, so as to monitor the patient's breathing. Sensors 6008 may also collect cardiac-related patient data in addition to respiratory-related patient data. In some cases, the implanted device 6000 may be in communication with other sensors, such as via a wireless communications protocol. Such sensors may include an external sensor 7000 (FIG. 1C), such as a pulse Doppler radar motion sensor, a pulse oximeter, a flow rate sensor, a pressure sensor, or other monitoring device coupled with sensors etc.

4.5 Data Management System

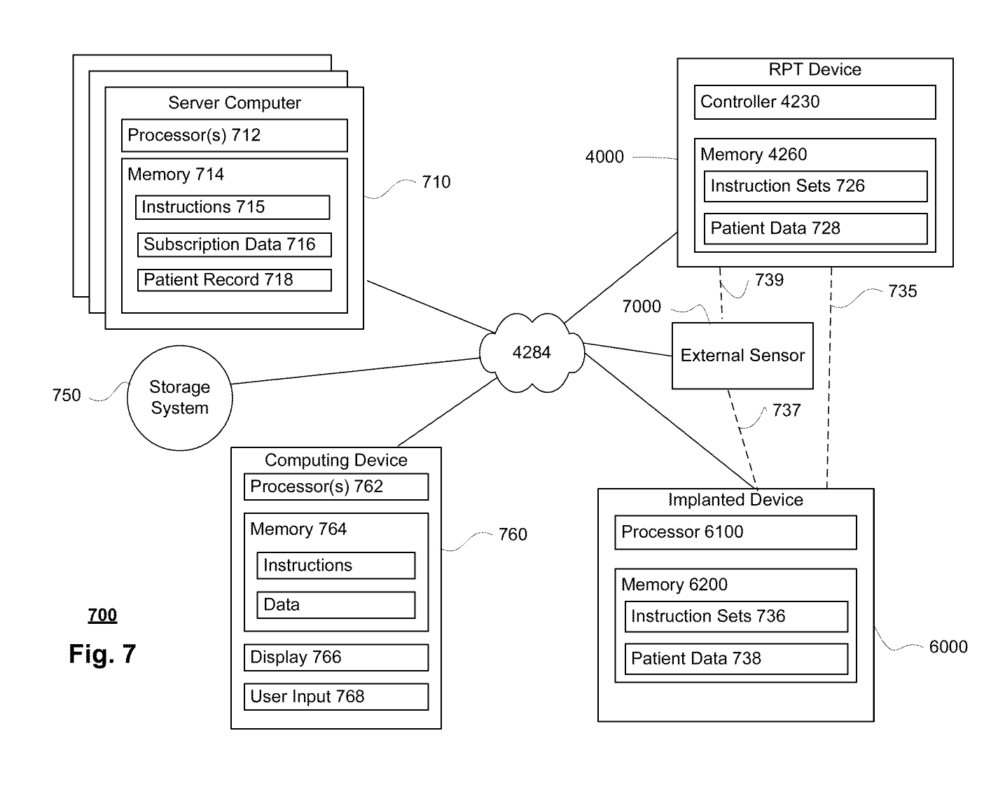

FIG. 7 depicts an example system 700 in which aspects of the technology of the present disclosure may be implemented. This example should not be considered as limiting the scope of the disclosure or usefulness of the features described herein. In this example, system 700 includes any one or more of server 710, RPT device 4000, implanted device 6000, external sensor 7000, storage system 750, as well as clinician computing device 760. These devices, if present, may each communicate, such as via a local communications network 4284 Implanted device 6000 may also communicate directly with RPT device 4000 via a wireless connection 735.

RPT device 4000 may include one or more components and work with other devices such as humidifier 5000 and patient interface 3000 (as shown in FIG. 1A). While only controller 4230 and memory 4260 are shown in FIG. 7, RPT device 4000 may include any of the components previously described. In addition, while RPT device 4000 is shown as communicating directly over network 4284, it may also communicate over network 4284 via an external computing device. For example, RPT device 4000 may communicate with a personal computer, smartphone, or other processing device that transmits data over network 4284.

Servers 710 may contain one or more processors 712, memory 714 and may be incorporated with other components typically present in general purpose computing devices. Memory 714 of server 710 may store information accessible by processor 712, including instructions 715 that can be executed by the processor 712. Memory 714 may also store data 718 that can be retrieved, manipulated or stored by processor 712. The memory can be of any non-transitory type capable of storing information accessible by the processor. The instructions 715 may include instructions that are directly or indirectly executed by processor 712. In that regard, the terms “instructions,” “application,” “steps” and “programs” can be used interchangeably herein. Functions, methods and routines of the instructions are explained in more detail below.

Patient record data 718 may be retrieved, stored or modified by processor 712 in accordance with the instructions 715. For instance, although the subject matter described herein is not limited by any particular data structure, the data can be stored in computer registers, in a relational database as a table having many different fields and records, or XML documents. Patient record data 718 may also be any information sufficient to identify or calculate relevant information, such as numbers, descriptive text, proprietary codes, pointers, references to data stored in other memories such as at other network locations. The one or more processors 712 may include conventional processors, such as a CPU, or may be a component, such as an ASIC.

Although FIG. 7 functionally illustrates the processor, memory, and other elements of servers 710, clinician computing device 760, RPT device 4000, and implanted device 6000 as each being within one block, the various components of each device may be stored within the different physical housings. For example, memory 714 may be a hard drive or other storage media located in a housing different from that of servers 710. Similarly, processor 712 may include a plurality of processors, some or all of which are located in a housing different from that of servers 710. Accordingly, references to a processor, computer, computing device, or memory will be understood to include references to a collection of processors, computers, computing devices, or memories that may or may not operate in parallel. Although some functions are described herein as taking place on a single computing device having a single processor, various aspects of the disclosure may be implemented by a plurality of computing devices communicating information with one another, such as by communicating over network 4284.

Network 4284 and intervening nodes described herein can be interconnected using various protocols and systems, such that the network can be part of the Internet, World Wide Web, specific intranets, wide area networks, local networks, or cell phone networks. The network can utilize standard communications protocols, such as Ethernet, Wi-Fi and HTTP, protocols that are proprietary to one or more companies, and various combinations of the foregoing. Although certain advantages are obtained when information is transmitted or received as noted above, other aspects of the subject matter described herein are not limited to any particular manner of transmission of information.

Servers 710 may include one or more communication servers that are capable of communicating with storage system 750, clinician computing device 760, RPT device 4000, and implanted device 6000 via network 4284. As will be described in greater detail below, servers 710 may transmit subscriptions over network 4284 to RPT device 4000 and implanted device 6000. In turn, RPT device 4000 and implanted device 6000 may transmit data to server 780 in accordance with the received subscriptions.

Clinician computing device 760 may be configured similarly to servers 710, with one or more processors 762 and memory 764 storing instructions as described above. Each clinician device 760 may be a personal computing device intended for use by a clinician and have all of the components normally used in connection with a personal computing device such as a central processing unit (CPU), memory (e.g., RAM and internal hard drives) storing data and instructions, a display such as a display 766 (e.g., a monitor having a screen, a touch-screen, a projector, a television, or other device that is operable to display information), and user input device 768 (e.g., a mouse, keyboard, touch-screen or microphone).

System 700 may also include external sensor 7000. External sensor 7000 may be a non-contact bed-side monitor as illustrated in FIG. 1C, such as the SleepMinder™ sensor, which utilizes Doppler radar to monitor the patient's condition. The SleepMinder sensor is described in U.S. Patent Application Pub. No. 200910203972, filed on Nov. 28, 2006, the disclosure of which is hereby incorporated by reference in its entirety. As shown in FIG. 7, external sensor 7000 may have a connection 739 with RPT device 4000 and nay have a connection 737 with implanted device 6000. These connections 739 and 737 may be wireless connections.

4.6 Example Methodologies

In accordance with one aspect of the disclosure, implanted device 6000 of FIG. 7 may use one or more sensors 6008 to collect cardiac-related patient data (e.g. patient's pulse, blood pressure, blood flow, ventricular contractions, heart rate, ECG, central venous pressure, temperature, peripheral venous oxygen saturation, blood gas, etc.)) and/or respiratory-related patient data representing one or more respiratory parameters (e.g., breathing rate, oximetry data, pneumatic or Respiratory Inductance Plethysmography (RIP)-based respiratory effort data, respiratory flow data, SDB events (e.g., central or obstructive apnea, central and/or obstructive hypopnea, Cheyne-Stokes respiration, and/or snoring, etc.)). This cardiac-related and respiratory-related patient data may be stored by implanted device 6000 as patient data 738 in a memory 6200. Implanted device 6000 may then transmit the stored patient data 738 to server 710 or other processing device, wherein the received patient data is associated with a particular implanted device 6000 and patient 1000. The received patient data 738 may then be stored as patient record 718. A clinician may then access the stored patient record 718 so as to determine the efficacy of the patient's cardiac treatment. For example, a user, such as a clinician, may use computing device 760 to request that server 710 provide patient record 718 for a particular patient. The patient may be identified in any number of ways, including by name or by identification number. The stored patient record 718 for the identified patient may then be transmitted to computing device 760, where it is analyzed to determine the efficacy of the patient's cardiac treatment. Server 710 may also contain instructions 715 wherein the analysis of a patient record 718 may be performed. For example, server 710 may transmit a report to computing device 760 indicating the efficacy of a patient's cardiac treatment.

RPT device 4000 may also be used to collect respiratory-related patient data, such as the patient's pulse oximetry, breathing rate, respiratory airflow rate, SDB events (e.g., central or obstructive apnea, central and/or obstructive hypopnea, Cheyne-Stokes respiration, snoring and/or flow limitation etc.) and respiratory effort data. The respiratory-related patient data collected by respiratory device 4000 may be stored as patient data 728 and transmitted to server 710 from RPT device 4000 in the same manner as patient data 738. RPT device 4000 and implanted device 6000 may also transfer patient data 728/738 directly to one another over wireless connection 735.

The collection of patient data by RPT device 4000 and implanted device 6000 may occur in accordance with instruction sets 726 and 736, respectively. Implanted device 6000 may implement instruction sets 736 using processor 6100, while RPT device 4000 may implement instruction sets 726 using controller 4230. These instruction sets 726 and 736 may each identify the type of patient data to be collected by RPT device 4000 and implanted device 6000, as well as the manner in which the patient data is to be collected. In particular, RPT device 4000 and implanted device 6000 may identify whether one or more conditions are met when patient data is being collected. For example, implanted device 6000 may determine whether a particular set of patient data 738 has been collected during a period in which the patient has maintained some predetermined baseline regarding the patient's cardiac and respiratory condition.

For example, as described above, a patient's SDB may contribute to changes or fluctuations in the cardiac-related and/or respiratory-related patient data collected by RPT device 4000 and/or implanted device 6000. These fluctuations may interfere with an efficacy determination, such as how well a particular cardiac treatment (e.g., a cardiac drug treatment) is working in treating the patient's cardiac condition (e.g. heart failure). For example, hemodynamic fluctuations, and other physical perturbations attributed to SDB, may interfere with a hemodynamic assessment or cardiac assessment by which the efficacy of a cardiac drug can be determined Thus, in accordance with one aspect, the disclosed system may detect the patient's SDB so as to establish a smooth hemodynamic baseline (i.e., cardiac-related patient data over time that is isolated from SDB events) so that changes to the baseline may be assessed, such as by server 710, in a manner isolated from an impact of SDB events. Evaluations of such an SDB-isolated baseline (e.g., by determining or calculating changes to the SDB-isolated baseline over a period) may then be more indicative of efficacy of a cardiac treatment implemented during such a period. In order to establish such an SDB-isolated baseline, any device of the system 700, such as implanted device 6000 or RPT device 4000, may measure or detect various attributes of the patient's respiratory condition to determine if they are within an acceptable range (e.g., detect one or more SDB events such as from respiratory-related patient data signals or the absence thereof such as from a flow rate signal from a flow rate sensor or motion sensor; or detection of other respiration issues such as from blood gas evaluation). The acceptable range and identification of the particular attributes may be based on whether they affect the smooth hemodynamic baseline that is suitable for determining the efficacy of a particular cardiac treatment.

For example, implanted device 6000 or RPT device 4000 may measure or receive a measure of the patient's oximetry levels to assess whether the patient's arterial blood has an oxygenation level that satisfies a threshold amount or is in a suitable range (e.g. at least 95%). Similarly, implanted device 6000 or RPT device 4000 may determine whether the patient's carbon dioxide level is within some predetermined threshold or threshold range. The implanted device 6000 or RPT device 4000 may also detect SDB events.

The implanted device 6000 or RPT device 4000 may also measure or receive a measure of one or more hemodynamic parameters of a patient, such as for determining that the parameter satisfies a threshold amount or is in a suitable range. For example, the device may receive pulmonary artery pressure or blood flow by a sensor such as an implanted pressure or flow sensor or a blood pressure cuff. Such changes may be indicative of the efficacy of a cardiac treatment. However, if the patient experiences some form of SDB, which may be detected by any device, the patient's hemodynamic parameters may change due to the SDB.

Accordingly, in some cases, an implanted device 6000 and/or RPT device 4000 may determine that an SDB event has occurred or that some other respiratory parameter deficiency exists. In response, the RPT device 4000 and/or implanted device 6000 may perform one or more actions to treat the patient's SDB (e.g., alleviate the SDB event such as by restoring normal breathing from detected Cheyne-Stokes respiration, apnea, hypopnea, etc.) which may in turn restore hemodynamic parameters to a level unaffected by the SDB event(s) (i.e., the SDB-isolated baseline). In particular, implanted device 6000 may provide genioglossal nerve stimulation to address a patient's OSA or phrenic nerve stimulation to address a patient's CSA. Similarly, RPT device 4000 may adjust the manner in which the respiratory pressure therapy is being provided to the patient. For example, RPT device 4000 may increase the CPAP treatment pressure, change a target ventilation measure for servo-ventilation therapy, change a Positive End Expiratory Pressure and/or adjust a setting of a pressure support ventilation therapy being supplied through patient interface 3000. RPT device 4000 may also respond by going from a standby state, in which little or no treatment pressure is being provided to the patient, to an active state, in which some predetermined treatment pressure or pressure support is provided to the patient. In some cases, a continuous positive airway pressure (CPAP) therapy may be discontinued in favor of a pressure support ventilation therapy. These SDB therapies may optionally be initiated upon receipt of SDB-related patient data from the different devices. For example, implanted device 6000 may transmit a signal to RPT device 4000 indicating information associated with SDB has occurred to trigger respiratory pressure therapy. Similarly, RPT device 4000 may transmit a signal to implanted device 6000 indicating information associated with SDB has occurred to trigger an implanted device 6000 therapy.

During the detection of the presence of such SDB events or some other respiratory parameter deficiency (i.e., when breathing is not normal), recorded data representing hemodynamic parameters over time may be optionally marked for their corresponding association with the SDB event(s) so as to permit their exclusion from determination of the efficacy of the cardiac treatment. Thus, a processor, for example, performing an assessment of positive or negative changes to hemodynamic parameters over time may readily disregard or separate those changes potentially attributable to SDB from those isolated from SDB. As such, an absence of SDB events or their controlled alleviation may then be identified. In one such example, a process may be implemented to detect certain changes in hemodynamic parameters to generate a report of the efficacy of a cardiac treatment. Such a report may show hemodynamic parameters (and/or their changes over time such as by calculating/determining trends in the data) using only data of such parameters that do not coincide in time with detected SDB events or untreated SDB events. Thus, the processor may disregard changes in hemodynamic parameters that are correlated (e.g., time or time period related) with detected SDB events and focus on an analysis of hemodynamic parameters that are isolated from (e.g., substantially out of time or time period synchronization with) SDB events. In such a way, decisions as to the effect of a cardiac treatment on hemodynamic parameters may be more accurately made.

In treating a patient's SDB so as to maintain such a smooth hemodynamic baseline isolated from effects of untreated SDB events, RPT device 4000 may provide advanced respiratory pressure therapies, such as adaptive servo-ventilation (ASV). Such ASV is a form of servo-ventilation that has a changeable, rather than fixed target ventilation. The changeable target ventilation may be learned from some characteristic of the patient, for example, a respiratory characteristic of the patient. Example implementations of ASV are described in PCT Patent Publication No. WO 2013/152403, the entire disclosure of which is herein incorporated by reference. In some such cases of ASV, a pressure waveform may be delivered to the patient. This pressure waveform may have an amplitude that is adjusted in reaction to the patient's breathing. By implementing ASV, RPT device 4000 may maintain various respiratory conditions within predefined thresholds, thereby helping to enforce the smooth hemodynamic baseline.

As described above, RPT device 4000 may receive cardiac-related and/or respiratory-related patient data from implanted device 6000. Similarly, implanted device 6000 may receive cardiac-related and/or respiratory-related patient data from RPT device 4000. In addition, RPT device 4000 and/or implanted device 6000 may receive respiratory-related and/or cardiac-related patient data from external sensor 7000, such as a stand-alone monitor. In one example, external sensor 7000 may be a SleepMinder sensor, which may monitor the patient's cardiac and/or respiratory condition with a Doppler radar motion sensor. RPT device 4000 and/or implanted device 6000 may receive Doppler radar motion data from external sensor 7000 and analyze the data to evaluate the patient's respiratory and cardiac conditions. Optionally, RPT device 4000 may then implement or adjust control parameters of respiratory pressure therapy in a manner that is based on the respiratory-related and/or cardiac-related patient data received from external sensor 7000. In particular, RPT device 4000 may analyze the received patient data to determine whether the patient's respiratory condition has experienced a change in state, whether it be an improved state or a worsening state. In response to the determined conditions, RPT device 4000 may adjust control parameters of the respiratory pressure therapy provided to the patient. Also optionally, implanted device 6000 may implement or adjust control parameters of a nerve stimulation therapy in a manner that is based on the respiratory-related and/or cardiac-related patient data received from external sensor 7000. In this way, RPT device 4000 and/or implanted device 6000 may monitor the patient's respiratory condition and treat the patient's SDB in a manner that may assist in stabilizing the patient's respiratory condition so as to achieve the smooth hemodynamic baseline.

In accordance with one aspect, implanted device 6000 and RPT device 4000 may be remotely programmed with predetermined thresholds. For example, a clinician may use computing device 760 to instruct sever 710 to transmit subscription data 716 to RPT device 4000, implanted device 6000, or both. The transmitted subscription data 716 may contain instructions to measure one or more patient conditions or detect certain changes or states of measurable parameters such as to identify whether each condition or measureable parameter satisfies some predefined threshold. The subscription data 716 may also contain instructions regarding how RPT device 4000 or implanted device 6000 should react if one or more of the identified conditions are determined to be outside of the predetermined thresholds. RPT device 4000 may store the received subscription data 716 as instruction sets 726, while implanted device 6000 may store the received subscription data 716 as instruction sets 736.

As described above, implanted device 6000 may provide a combination of cardiac and respiratory therapy. In one example, the patient may receive both cardiac and respiratory therapy from implanted device 6000 without the use of RPT device 4000, humidifier 5000, and patient interface 3000. For example, implanted device 6000 may provide the patient with cardiac resynchronization therapy to address one or more cardiac conditions, while also providing the patient with respiratory therapy in the form of genioglossal nerve stimulation for OSA and phrenic nerve stimulation for CSA. In this way, cardiac treatment and respiratory therapy may be provided to the patient through a single implanted device 6000.

In another example, RPT device 4000 may operate in cooperation with implanted device 6000. In particular, implanted device 6000 may provide cardiac treatment, while RPT device provides respiratory pressure therapy. In still another example, RPT device 4000 and implanted device 6000 may both provide some form of respiratory therapy. In this example, implanted device 6000 may provide some form of nerve stimulation in order to alleviate the patient's SDB, however if the nerve stimulation remains or becomes ineffective for some reason, RPT device 4000 may be triggered to provide supplemental respiratory therapy such as a change in a respiratory pressure therapy (e.g., a change in control parameters of ASV). In this example, RPT device 4000 may remain in a standby mode until it is determined that a change in respiratory pressure therapy is needed.

In some cases, implanted device 6000 or RPT device 4000 may be implemented with one or more sensors to receive a measure of arterial pressure. For example, as shown in FIG. 1B, implanted device 6000 may include, or communicate with, sensor 6008 located within the patient's pulmonary arterial tree. In acquiring or measuring the patient's pulmonary arterial pressure and/or any other of the sensor data available to it, implanted device 6000 or RPT device 4000 may evaluate changes to predict cardiac events, such as acute decompensated heart failure, or to evaluate cardiac condition. In addition, the measured pulmonary arterial pressure and/or any other of the sensor data available to implanted device 6000 may also be evaluated for adjusting a cardiac treatment such as diuretics being administered to the patient. This adjustment is known as titration of the cardiac treatment. Thus, with examples of the present technology, diuretics may be titrated according to measured parameters while minimizing or eliminating the potential for SDB to interfere. Thus, implanted device 6000, such as with external sensor 7000 and/or RPT device 4000, may detect a patient's SDB and alleviate it by respiratory therapy or isolate titration-indicative measurements (e.g., pressure) from the SDB events so that the SDB-isolated measurements may be evaluated. Thus, a smooth hemodynamic, or SDB-isolated, baseline can be achieved for making adjustments to a diuretic dose in connection with the measured parameters (e.g., pulmonary arterial pressure and/or cardiac-related patient data (such as heart rate) such as those detected with a Doppler radar sensor and/or a pulmonary arterial sensor) which are unaffected by SDB. This may then permit a diuretic to be titrated more effectively.

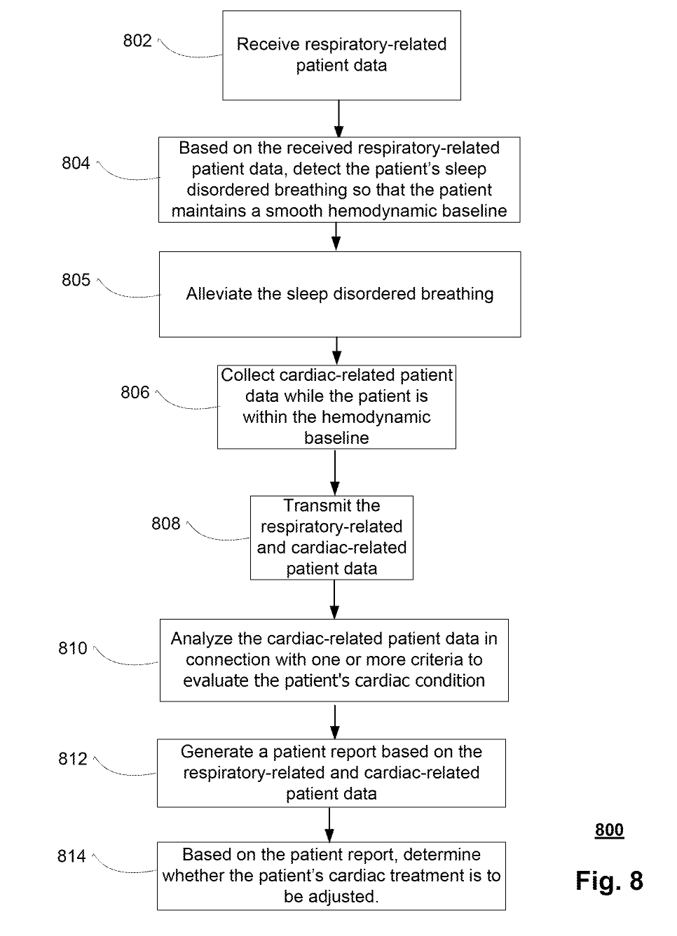

FIG. 8 is a flow diagram of a method 800 that may be performed by the disclosed system 700 described above. The method 800 may be performed in conjunction with a cardiac treatment of the patient. In block 802, the respiratory-related patient data is received. The respiratory-related patient data may be measured in one of the manners described above by RPT device 4000, implanted device 6000, external sensor 7000, or some combination thereof. Based on the received respiratory-related patient data, the patient's SDB may be detected or its absence confirmed so that the patient maintains an SDB-isolated baseline, such as a smooth hemodynamic baseline (block 804) of one or more measured hemodynamic parameters. If SDB is present or detected, respiratory therapy may optionally be performed or adjusted at step 805 by RPT device 4000, implanted device 6000, or some combination of the two in order to alleviate the SDB. For example, RPT device 4000 may apply ASV that is based on the patient's respiratory condition. Cardiac-related patient data may then be collected while the patient is within the SDB-isolated baseline (block 806). The cardiac-related patient data (e.g., hemodynamic parameters) may then be measured or analyzed based on the determined absence of SDB events in one of the manners described above by RPT device 4000, implanted device 6000, external sensor 7000, or some combination thereof.

In some cases, the respiratory-related and/or cardiac-related patient data may optionally be transmitted to a remote device, such as server 710 (block 808), where the respiratory-related and/or cardiac-related patient data may be analyzed in connection with one or more criteria to evaluate the patient's cardiac condition (block 810). If the method 800 is being performed in conjunction with a cardiac treatment of the patient, step 810 may include a determination of the efficacy of the patient's cardiac treatment. As discussed above, server 710 may determine, based on the cardiac-related patient data collected during the SDB-isolated baseline, whether the patient has experienced a change or not in one or more measured parameters that may be indicative of a response to the cardiac treatment. For example, an analysis of measured parameters may detect changes in any acute or chronic cardiac conditions over some predetermined period of time. In block 812, a patient report may be generated based on at least a portion of the cardiac-related patient data. The report may also be based on the respiratory-related patient data. Based on the patient's cardiac condition and/or the determined efficacy of the cardiac treatment, a determination may be made whether the patient's cardiac treatment is to be adjusted (block 814). This adjustment is known as titration of the cardiac treatment. In one example, server 710 may provide this determination by comparing the cardiac condition with a set of cardiac criteria. Titration may, for example, involve a change to a diuretic dose or other drug treatment or a change to a therapy such as a change in operation of CRT (e.g., a rhythm and/or amplitude pacing adjustment of the electrical stimulation therapy). In some cases, the titration of the cardiac treatment may involve an adjustment to a target ventilation of a respiratory pressure therapy, such as changing a tidal volume target or other volumetric target (e.g., minute ventilation target) where the ventilation target serves as a control in the delivery of Pressure Support (PS) ventilation therapy such as with an adaptive servo-ventilator of the system. Such titration changes may be identified (e.g., output to a display for human implementation) by a processor of the system or controlled by a processor for the system.

Operations may be added or removed from flow diagram 800. In addition, various operations need not be performed in the same order as set forth above.

4.7 Glossary

For the purposes of the present technology disclosure, in certain forms of the present technology, one or more of the following definitions may apply. In other forms of the present technology, alternative definitions may apply.

### 4.7.1 General

Air: In certain forms of the present technology, air may be taken to mean atmospheric air, and in other forms of the present technology air may be taken to mean some other combination of breathable gases, e.g. atmospheric air enriched with oxygen.

Respiratory Pressure Therapy (RPT): The application of a supply of air to an entrance to the airways at a treatment pressure that is continuously positive with respect to atmosphere.

Continuous Positive Airway Pressure (CPAP) therapy: Respiratory pressure therapy in which the treatment pressure is approximately constant through a respiratory cycle of a patient. In some forms of CPAP therapy, the pressure at the entrance to the airways will be slightly higher during exhalation, and slightly lower during inhalation. In some forms, the treatment pressure will vary between different respiratory cycles of the patient, for example, being increased in response to detection of indications of partial upper airway obstruction, and decreased in the absence of indications of partial upper airway obstruction.

Patient: A person, whether or not they are suffering from a respiratory or cardiac condition.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

### 4.7.2 Aspects of the Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea is said to have occurred when an apnea is detected that is due to a reduction in breathing effort, or the absence of breathing effort, despite the airway being patent. A mixed apnea is said to have occurred when a reduction or absence of breathing effort coincides with an obstructed airway.

Breathing rate: The rate of spontaneous respiration of a patient, usually measured in breaths per minute.

Duty cycle: The ratio of inhalation time, Ti to total breath time, Ttot.

Effort (breathing): Breathing effort will be said to be the work done by a spontaneously breathing person attempting to breathe.

Expiratory portion of a breathing cycle: The period from the start of expiratory flow to the start of inspiratory flow.

Flow limitation: Flow limitation will be taken to be the state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Types of flow limited inspiratory waveforms:

(i) Flattened: Having a rise followed by a relatively flat portion, followed by a fall.

(ii) M-shaped: Having two local peaks, one at the leading edge, and one at the trailing edge, and a relatively flat portion between the two peaks.

(iii) Chair-shaped: Having a single local peak, the peak being at the leading edge, followed by a relatively flat portion.

(iv) Reverse-chair shaped: Having a relatively flat portion followed by single local peak, the peak being at the trailing edge.

Hypopnea: A reduction in flow, but not a cessation of flow. In one form, a hypopnea may be said to have occurred when there is a reduction in flow below a threshold for a duration. A central hypopnea is said to have occurred when a hypopnea is detected that is due to a reduction in breathing effort. In one form in adults, either of the following may be regarded as being hypopneas:

(i) a 30% reduction in patient breathing for at least 10 seconds plus an associated 4% desaturation; or

(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed (obstructed).

Positive End-Expiratory Pressure (PEEP): The pressure above atmosphere in the lungs that exists at the end of expiration.

Peak flow rate (Qpeak): The maximum value of flow rate during the inspiratory portion of the respiratory flow waveform.

Respiratory flow rate, airflow rate, patient airflow rate, respiratory airflow rate (Qr): These synonymous terms may be understood to refer to the RPT device's estimate of respiratory airflow rate, as opposed to “true respiratory flow rate” or “true respiratory airflow rate”, which is the actual respiratory flow rate experienced by the patient, usually expressed in litres per minute.

Tidal volume (Vt): The volume of air inhaled or exhaled during normal breathing, when extra effort is not applied.

Inspiratory (inhalation) Time (Ti): The duration of the inspiratory portion of the respiratory flow rate waveform.

Expiratory (exhalation) Time (Te): The duration of the expiratory portion of the respiratory flow rate waveform.

Respiratory (total) Time (Ttot): The total duration between the start of the inspiratory portion of one respiratory flow rate waveform and the start of the inspiratory portion of the following respiratory flow rate waveform.

Typical recent ventilation: The value of ventilation around which recent values over some predetermined timescale tend to cluster, that is, a measure of the central tendency of the recent values of ventilation.

Upper airway obstruction (UAO): includes both partial and total upper airway obstruction. This may be associated with a state of flow limitation, in which the level of flow increases only slightly or may even decrease as the pressure difference across the upper airway increases (Starling resistor behaviour).

Ventilation (Vent): A measure of the total amount of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 4.7.3 RPT Device Parameters

Flow rate: The instantaneous volume (or mass) of air delivered per unit time. While flow rate and ventilation have the same dimensions of volume or mass per unit time, flow rate is measured over a much shorter period of time. In some cases, a reference to flow rate will be a reference to a scalar quantity, namely a quantity having magnitude only. In other cases, a reference to flow rate will be a reference to a vector quantity, namely a quantity having both magnitude and direction. Where it is referred to as a signed quantity, a flow rate may be nominally positive for the inspiratory portion of a breathing cycle of a patient, and hence negative for the expiratory portion of the breathing cycle of a patient. Flow rate will be given the symbol Q. ‘Flow rate’ is sometimes shortened to simply ‘flow’. Total flow rate, Qt, is the flow rate of air leaving the RPT device. Vent flow rate, Qv, is the flow rate of air leaving a vent to allow washout of exhaled gases. Leak flow rate, Ql, is the flow rate of leak from a patient interface system. Respiratory flow rate, Qr, is the flow rate of air that is received into the patient's respiratory system.

Leak: An unintended flow of air. In one example, leak may occur as the result of an incomplete seal between a mask and a patient's face. In another example leak may occur in a swivel elbow to the ambient.

### 4.7.4 Terms for Ventilators

Adaptive Servo-Ventilator (ASV): A servo-ventilator that has a changeable, rather than fixed target ventilation. The changeable target ventilation may be learned from some characteristic of the patient, for example, a respiratory characteristic of the patient.

Backup rate: A parameter of a ventilator that establishes the minimum breathing rate (typically in number of breaths per minute) that the ventilator will deliver to the patient, if not triggered by spontaneous respiratory effort.

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

Expiratory positive airway pressure (EPAP): a base pressure, to which a pressure varying within the breath is added to produce the desired mask pressure which the ventilator will attempt to achieve at a given time.

Inspiratory positive airway pressure (IPAP): Maximum desired mask pressure which the ventilator will attempt to achieve during the inspiratory portion of the breath.

Pressure Support: A value that is indicative of the increase in pressure during ventilator inspiration over that during ventilator expiration, and generally means the difference in pressure between the maximum value during inspiration and the minimum value during expiration (e.g., PS=IPAP−EPAP). In some contexts pressure support means the difference which the ventilator aims to achieve, rather than what it actually achieves.

Servo-ventilator: A ventilator that measures patient ventilation, has a target ventilation, and which adjusts the level of pressure support to bring the patient ventilation towards the target ventilation.

Spontaneous/Timed (S/T): A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator delivers a breath of air to a spontaneously breathing patient, it is said to be triggered to do so at the initiation of the respiratory portion of the breathing cycle by the patient's efforts.

Typical recent ventilation (Vtyp): The value around which recent measures of ventilation over some predetermined timescale tend to cluster, that is, a measure of the central tendency of the measures of ventilation over recent history.

Ventilator: A mechanical device that provides pressure support to a patient to perform some or all of the work of breathing.

4.8 Other Remarks

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being preferably used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated by reference to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

Moreover, in interpreting the disclosure, all terms should be interpreted in the broadest reasonable manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular embodiments, it is to be understood that these embodiments are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative embodiments and that other arrangements may be devised without departing from the spirit and scope of the technology.

### 5 REFERENCE LABEL LIST

system 700
server 710
processors 712
memory 714
instructions 715
subscription data 716
patient record 718
instruction sets 726
patient data 728
wireless connection 735
instruction sets 736
connection 737
patient data 738
connection 739
storage system 750
computing device 760
processors 762
memory 764
display 766
user input device 768
server 780
method 800
block 802
block 804
step 805
block 806
block 808
block 810
block 812
block 814
patient 1000
patient's heart 1002
patient interface 3000
seal-forming structure 3100
plenum chamber 3200
structure 3300
vent 3400
connection port 3600
forehead support 3700
RPT device 4000
external housing 4010
upper portion 4012
portion 4014
panel 4015
chassis 4016
handle 4018
pneumatic block 4020
pneumatic components 4100
air filter 4110
inlet air filter 4112
outlet air filter 4114
muffler 4120
inlet muffler 4122
outlet muffler 4124
pressure generator 4140
controllable blower 4142
motor 4144
anti-spill back valve 4160
air circuit 4170
supplemental oxygen 4180
electrical components 4200
PCBA 4202
power supply 4210
input devices 4220
central controller 4230
clock 4232
therapy device controller 4240
protection circuit 4250
memory 4260
transducers 4270
pressure sensor 4272
flow rate sensor 4274
motor speed transducer 4276
data communication interface 4280
network 4282
network 4284
remote external device 4286
local external device 4288
output device 4290
display driver 4292
display 4294
pressure control module 4330
humidifier 5000
humidifier controller 5250
implanted device 6000
lead wire 6002
electrode 6003
lead wire 6004
electrode 6005
wires 6006
sensors 6008
electrodes 6009
processor 6100
memory 6200
external sensor 7000

1. A method for evaluating a cardiac condition of a patient with sleep disordered breathing comprising:
receiving data representing one or more respiratory parameters for the patient;
receiving cardiac-related patient data for the patient;
determining in a processor a presence of a smooth hemodynamic baseline from the received data representing one or more respiratory parameters for the patient; and
evaluating, with the processor, the cardiac condition of the patient from the received cardiac-related patient data based on the determined presence of the smooth hemodynamic baseline.

2. The method of claim 1 wherein the determining comprises detecting absence of sleep disordered breathing events for a predetermined period of time.

3. The method of claim 1 wherein the determining comprises:
detecting sleep disordered breathing events; and
controlling a respiratory therapy to alleviate the detected sleep disordered breathing events for a predetermined period of time.

4. The method of claim 1 further comprising collecting, with one or more sensors, data representing one or more hemodynamic parameters for the patient while the patient is within the smooth hemodynamic baseline.

5. The method of claim 4, wherein the evaluating is based on the data representing one or more hemodynamic parameters.

6. The method of claim 3 wherein the controlling comprises controlling a respiratory pressure therapy device to deliver respiratory pressure therapy for the patient.

7. The method of claim 6, wherein the respiratory pressure therapy comprises an adaptive servo-ventilation therapy.

8. The method of claim 2 wherein the sleep disordered breathing events comprise one or more of apnea, hypopnea, Cheyne-Stokes respiration, snoring and flow limitation.

9. The method of claim 3, wherein the controlling comprises controlling an implanted device to provide at least one of genioglossal nerve stimulation and phrenic nerve stimulation.

10. The method of claim 1, further comprising determining, based on the evaluating, efficacy of a cardiac treatment that has been provided to the patient.

11. The method of claim 10, wherein the cardiac treatment comprises one or more of administering a pharmaceutical cardiac drug and application of a cardiac resynchronization therapy.

12. The method of claim 10, further comprising titrating the cardiac treatment according to the cardiac condition of the patient.

13. The method of claim 12 wherein the titration of the cardiac treatment comprises determination of a diuretic dose.

14. The method of claim 12 wherein the titration of the cardiac treatment comprises an adjustment to a target ventilation of a respiratory pressure therapy.

15. The method of claim 14 wherein the respiratory pressure therapy is a pressure support ventilation therapy controlled in accordance with the target ventilation.

16. The method of claim 5 wherein the evaluating comprises outputting one or more hemodynamic parameters according to a time period of the smooth hemodynamic baseline.

17. The method of claim 5 wherein the evaluating comprises detecting a change in one or more hemodynamic parameters within a time period initiated with the smooth hemodynamic baseline.

18. The method of claim 1 wherein the received cardiac-related patient data comprises measurements generated by a pulmonary artery sensor.

19. The method of claim 1 wherein the received data representing one or more respiratory parameters comprises measurements generated by a Doppler radar motion sensor.

20. A system for evaluating a cardiac condition of a patient with sleep disordered breathing comprising:
one or more memories; and
one or more processors in communication with the one or more memories, the one or more processors configured to:
receive data representing one or more respiratory parameters for the patient;
receive cardiac-related patient data for the patient;
determine a presence of a smooth hemodynamic baseline from the received data representing one or more respiratory parameters for the patient; and
evaluate the cardiac condition of the patient from the received cardiac-related patient data for the patient based on the determined presence of the smooth hemodynamic baseline.

21. The system of claim 20 wherein determination of the presence of the smooth hemodynamic baseline comprises detecting absence of sleep disordered breathing events for a predetermined period of time.

22. The system of claim 20 wherein determination of the presence of the smooth hemodynamic baseline comprises detecting sleep disordered breathing events and controlling a respiratory therapy to alleviate the detected sleep disordered breathing events for a predetermined period of time.

23. The system of claim 20 further comprising collecting, with one or more sensors, cardiac-related patient data while the patient is within the smooth hemodynamic baseline.

24. The system of claim 23 wherein evaluation of the cardiac condition is based on the cardiac-related patient data.

25. The system of claim 20 further comprising a respiratory pressure therapy apparatus, the apparatus configured to alleviate detected sleep disordered breathing events by controlling a respiratory pressure therapy for the patient.

26. The system of claim 25, wherein the respiratory pressure therapy comprises an adaptive servo-ventilation therapy.

27. The system of claim 21 wherein the sleep disordered breathing events comprise one or more of apneas, hypopneas, Cheyne-Stokes respiration, snoring, and flow limitation.

28. The system of claim 20, further comprising an implanted device configured to alleviate sleep disordered breathing events by controlling providing at least one of genioglossal nerve stimulation and phrenic nerve stimulation.

29. The system of claim 20, wherein the one or more processors is further configured to determine, based on the evaluating, efficacy of a cardiac treatment that has been provided to the patient.

30. The system of claim 29, wherein the cardiac treatment comprises one or more of administering a pharmaceutical cardiac drug and providing a cardiac resynchronization therapy.

31. The system of claim 29, wherein the evaluation further comprises titration of the cardiac treatment according to the cardiac condition of the patient.

32. The system of claim 31 wherein the cardiac treatment comprises a diuretic dose and the titration comprises determining the diuretic dose.

33. The system of claim 31 wherein the titration of the cardiac treatment comprises an adjustment to a target ventilation of a respiratory pressure therapy.

34. The system of claim 33 wherein the respiratory pressure therapy is a pressure support ventilation therapy controlled in accordance with the target ventilation and provided by an adaptive servo-ventilator of the system.

35. The system of claim 20 wherein the evaluation comprises outputting, by the one or more processors, one or more hemodynamic parameters according to a time period of the smooth hemodynamic baseline.

36. The system of claim 20 wherein the evaluation comprises detecting by the one or more processors a change in one or more hemodynamic parameters within a time period initiated with the smooth hemodynamic baseline.

37. The system of claim 20 wherein the received cardiac-related patient data comprises measurements generated by a pulmonary artery sensor.

38. The system of claim 20 wherein the received data representing one or more respiratory parameters comprises measurements generated by a Doppler radar motion sensor.

39. An apparatus for evaluating a cardiac condition of a patient with sleep disordered breathing comprising:
a flow generator for providing a supply of air to a patient interface at variable pressures, the flow generator adapted for coupling with a patient interface for delivering the supply of air to an entrance of an airway of the patient;
one or more sensors configured to sense one or more respiratory parameters for the patient; and
one or more processors, the one or more processors configured to:
control the supply of air at variable pressures;
receive data representing the one or more respiratory parameters for a patient; and
determine a presence of a smooth hemodynamic baseline from the received data representing the one or more respiratory parameters for the patient.

40. The apparatus of claim 39 further comprising:
one or more sensors configured to sense one or more hemodynamic parameters for the patient;
wherein the one or more processors is further configured to:
receive data representing one or more hemodynamic parameters for the patient; and
evaluate the cardiac condition of the patient from the received data representing one or more hemodynamic parameters for the patient based on the determined presence of the smooth hemodynamic baseline.

41. The apparatus of claim 39, wherein the one or more processors are further configured to receive a transmission from an implanted device.

42. The apparatus of claim 39, wherein the one or more processors are further configured to receive a transmission from a Doppler radar motion sensor.

43. The apparatus of claim 41, wherein the one or more processors are further configured to change a setting for control of the supply of air based on the received transmission.

44. The apparatus of claim 39, wherein control of the supply of air at variable pressures comprises adaptive servo-ventilation.

45. A method for evaluating a cardiac condition of a patient with sleep disordered breathing comprising:
controlling a flow generator to provide a supply of air to a patient interface, the flow generator adapted for coupling with a patient interface for delivering the supply of air to an entrance of an airway of the patient;
sensing, with one or more sensors, one or more respiratory parameters for the patient; and
in one or more processors:
controlling the supply of air at variable pressures;
receiving data representing the one or more respiratory parameters for a patient; and
determining a presence of a smooth hemodynamic baseline from the received data representing the one or more respiratory parameters for the patient.

46. The method of claim 45 further comprising:
sensing, with one or more sensors, one or more hemodynamic parameters for the patient; and
in the one or more processors:
receiving data representing one or more hemodynamic parameters for the patient; and
evaluating the cardiac condition of the patient from the received data representing one or more hemodynamic parameters for the patient based on the determined presence of the smooth hemodynamic baseline.

47. The method of claim 45, further comprising, with the one or more processors, receiving a transmission from an implanted device.

48. The method of claim 47, further comprising, with the one or more processors, changing a setting for control of the supply of air based on the received transmission.

49. The method of claim 48, wherein the controlling the supply of air at variable pressures comprises adaptive servo-ventilation.

50. The method of claim 45, further comprising, with the one or more processors, receiving a transmission from a Doppler radar motion sensor.

51. The method of claim 50, further comprising, with the one or more processors, changing a setting for control of the supply of air based on the received transmission.

52. The method of claim 51, wherein the controlling the supply of air at variable pressures comprises adaptive servo-ventilation.
